(19) United States
(12) Patent Application Publication
(10) Pub. No.: US 2007/0238808 A1
(43) Pub. Date: Oct. 11, 2007

(54) DENTAL MATERIALS, METHODS OF MAKING AND USING THE SAME, AND ARTICLES FORMED THEREFROM

(76) Inventors: A. Jon Goldberg, West Hartford, CT (US); Patrick T. Mather, Chagrin Falls, OH (US); Thomas K. Wood, College Station, TX (US)

Correspondence Address: CANTOR COLBURN, LLP, 55 GRIFFIN ROAD SOUTH, BLOOMFIELD, CT, 06002, US

(21) Appl. No.: 11/684,314

(22) Filed: Mar. 9, 2007

Related U.S. Application Data
(60) Provisional application No. 60/780,766, filed on Mar. 9, 2006.

(51) Int. Cl. A61K 6/08

(52) U.S. Cl. 523/116

(57) ABSTRACT

Disclosed herein is a dental material composition comprising a polymer matrix material and a biocatalyst that promotes the deposition of a mineral, for example silica or hydroxylapatite. A dental restoration comprising the polymer matrix material and biocatalyst is also claimed. In another embodiment, a method of forming a dental material comprises combining a biocatalyst that promotes the deposition of a mineral and a polymer matrix material to form a biocatalyst-polymer composite.

### CROSS-REFERENCE TO RELATED APPLICATIONS

This application claims the benefit of U.S. Patent Application Ser. No. 60/780,766, filed Mar. 9, 2006, which is incorporated by reference herein in its entirety.

### BACKGROUND

This invention relates to dental materials, and in particular to new dental materials containing a polymeric component and a ceramic component, methods for the manufacture and use thereof, as well as articles made therefrom.

Dental disease, injury, and anomalies can be treated by restoring or replacing the damaged or anomalous tissues with synthetic engineered materials. A wide variety of materials have been used or proposed for use, including metals and metal alloys, polymers (plastics), ceramics, and composites (combinations of two or more types of materials). It is generally recognized that none of the currently available dental restorative materials is ideal. For example, one drawback associated with current materials is that effective bonding and sealing to the tooth structure is difficult, and often results in microleakage between the restorative material and the tooth. This can lead to a recurrence of dental disease, requiring replacement, and possibly enlargement of the original restoration.

In addition, while each material has certain advantages, each also has certain disadvantages. For example, composites made from a polymer matrix and a particulate ceramic filler have improved mechanical properties compared to polymers alone. However, when the composites are used in restorative applications, the polymer exposed at the surface of the restoration tends to stain and absorb water. The surface also wears during normal biting and chewing, eventually exposing the ceramic particles. The particles can then themselves detach from the polymer matrix, thereby exposing new polymer, and continuing the wearing process. Even recently developed products exhibit approximately 25-35 micrometer (μm) of wear after one-year clinical trials.

The filler-matrix interface is also susceptible to deterioration, through hydrolysis of the silane coupling agent used to increase the bond between the filler and the polymer. Stress at the interface due to polymerization shrinkage might also be responsible for initiation of cracks. Accordingly, the silanation process is the subject of much study and attempts are being made to re-design the interphase region.

Improvements in polymer-ceramic composites have come about primarily by reducing particle size and increasing filler volume. These two factors simultaneously improve mechanical properties, surface luster, smoothness, and handling characteristics. Current “all-purpose” restorative composites typically contain 60-70 volume % filler, wherein the filler is a combination of particles 0.2-3.0 micrometers and 0.04 micrometers in diameter. Excessive amounts of filler can degrade the composite properties, however, and increase the viscosity of the composite to an unmanageable level, both from the standpoint of manufacture and use. For example, microfilled composites containing 0.04 micrometer fumed silica have been used, but the large surface area of the particles and resulting large particle-matrix interfacial area limits filler loadings to about 40% in order to maintain clinical handling characteristics.

Accordingly, there remains a continuing need in the art for new dental restorative materials that have improved properties, for example improved bonding to tooth structures, wear characteristics, more effective sealing, ease of use, and the like. It would further be advantageous if the materials were readily available and easy to manufacture.

### SUMMARY OF THE INVENTION

In one embodiment, a dental material composition comprises a polymer matrix material and a biocatalyst that promotes the deposition of a mineral, for example silica or a calcium phosphate from a mineral precursor. A dental restoration comprising the polymer matrix material and biocatalyst is also claimed.

In another embodiment, a method of forming a dental material comprises combining a biocatalyst that promotes the deposition of a mineral and a polymer matrix material to form a biocatalyst-polymer composite. The dental material can further comprise a mineral precursor.

In yet another embodiment, a dental treatment method comprises applying a biocatalyst-polymer composite to a dental site to be treated; and contacting the biocatalyst-polymer composite with a mineral precursor in an amount and for a time effective to deposit a mineral, forming a biocatalyst-polymer-mineral composite.

A Method of Forming a Dental Restoration Comprises Applying a Biocatalyst-polymer-mineral composite to a dental site to be restored.

The invention is further described by reference to the following figures and detailed description.

### BRIEF DESCRIPTION OF THE DRAWINGS

FIG. 1 is a schematic representation of DNA shuffling.

FIG. 2 is a schematic showing an electrospinning process.

FIG. 3 is a schematic diagram showing three different scaffolds

FIG. 4 is an electron micrograph of a poly(lactic acid) network.

FIGS. 5a-b are optical micrographs of precipitated silica.

FIGS. 6a-c are optical micrographs of precipitated silica.

FIGS. 7a-c illustrate formation of silica on poly(acrylate) films.

FIG. 8 is an electron micrograph of electrospun PVA/poly(lysine) fibers.

FIGS. 9a-c illustrate electrospun PVA/poly(lysine) fibers.

FIG. 10 illustrates photolithography of microlines.

FIG. 11 illustrates a microfluidic network.

FIG. 12 shows scanning electron micrographs of layer-by-layer films after application of TMOS precursor solution, wherein 12(a) shows no poly(L-lysine) (PLL) catalyst, and 12(b) shows 20 layers of PLL catalyst. The scale bars are each 45 micrometers long.

FIG. 13a shows the AFM topography of a silica particle formed on polymer/catalyst (PSS-PLL)₂₀ film and FIG. 13b is an AFM Phase image showing that the smooth and thick formed silica is constituted by nanosized particles (dark, small phase angle) embedded in a matrix of large phase angle. Inset scales are height (nm) and phase angle (deg), respectively, while lateral dimensions are shown in μm.

FIG. 14 illustrates FTIR reflectance spectra of silicified (PSS-PLL)₂₀ compared to a standard spectra of silica, confirming the formation of silica.

FIG. 15 is a graph illustrating modulus (GPa) vs. depth (nm) profiles for dendritic (▴) and less textured regions (●) of silica formed using PLL on quartz disc substrate. The modulus of the formed silica is 40-50 GPa, depending on morphology.

FIGS. 16a-b are two scanning electron micrographs of silica formed on dimethacrylate-based dental resin (Bond-1™), wherein 16a shows layers approximately 1 μm thick, and 16b shows angular particles that are submicron in size.

FIG. 17 is a canning electron micrograph of a cured composite silicified prior to cure.

FIG. 18 is a graph illustrating PLL-catalyzed silica condensation from silicic acid as measured by reactant depletion spectrophotometrically.

FIG. 19 is a graph illustrating residual silica yield (%) vs. concentration of PEI (wt %) profiles for TMOS exposed to PEI in solution.

FIG. 20 is a graph illustrating HA growth as a function of volume of PGA added (mL) vs. time (min) profiles for HA precursors exposed to PGA in solution.

FIG. 21 is a scanning electron micrograph of silica formed from TMOS on a single layer of PSS/PEI on a quartz disc.

FIG. 22 is a scanning electron micrograph of a mineral formed on a PEI-(PGA-PAH)₅-PGA multilayer film on a silicon wafer.

FIGS. 23a-f are scanning electron micrographs of silica formed from TMOS on multilayer films on silicon wafers.

FIGS. 24a-d are atomic force micrographs of silica catalyzed onto (PLL/PSS)₁₀ multilayer films from (a) 10 mM TMOS in buffer, (b) 200 mM TMOS in buffer, (c) 100 mM TMOS in ethanol and (d) non-catalyzed silica from 100 mM TMOS in ethanol.

FIGS. 25a-c are atomic force micrographs of (a) non-catalyzed silica dried at room temperature (RT), (b) catalyzed silica dried at RT, and (c) catalyzed silica dried at 150° C.

### DETAILED DESCRIPTION OF THE INVENTION

The inventors hereof have discovered a completely novel approach for incorporating a mineral filler into a polymeric matrix, to provide composites useful as dental materials. In particular, it has been discovered that certain biocatalysts, such as certain proteins or polyamines capable of catalyzing the deposition of minerals, for example, silica or calcium phosphates such as hydroxylapatite (HA), can remain functional in a polymer matrix, and that such biocatalyst-polymer compositions can be used in various restorative dental applications. Incorporation of mineral materials such as ceramic in this manner, i.e., enzymatic deposition, avoids many of the drawbacks associated with manufacture of traditional ceramic-polymer composites, and provides novel materials with new properties.

“Dental materials” as used herein refers to materials used in dentistry for a variety of therapeutic purposes, including but not limited to restorative materials, bonding agents, sealants, desensitization agents, implants, orthodontic wires, brackets, posts, endodontic sealants, repair of bone defects, bone growth around dental implants, and the like.

In one embodiment the novel dental material comprises a polymer matrix and a mineral-rich surface layer with high hardness and excellent wear resistance. In accordance with the invention, a polymer matrix that contains or is coated with a mineral-depositing biocatalyst is contacted with a mineral precursor, whereby the mineral (for example, a ceramic such as silica or a calcium phosphate such as hydroxylapatite) is deposited within and/or on the polymer matrix material. A “mineral” as used herein refers to any inorganic material. A preferred class of minerals is ceramics, that is, inorganic materials that do not contain a metal. “Deposited” and “deposition” as used herein refers to the biocatalyzed formation of a mineral from a mineral precursor in the presence of a polymer. For example, “deposited” as applied to silica (SiO₂) refers to the hydrolysis and condensation of silica precursor molecules (for example, tetraethyl orthosilicate (TEOS) or tetramethyl orthosilicate (TMOS)), resulting in solid SiOH/SiO₂ at the surface of and/or within the polymer matrix. In one embodiment, a surface layer of mineral-rich polymer is formed in situ following conventional chairside or laboratory dental procedures.

The surface layer is obtained by enzymatic formation of the mineral under ambient conditions using natural or engineered enzymes immobilized within the polymeric component. The term “biocatalyst” is used herein to refer to those proteins or small molecules that promote the deposition of a mineral. It is to be understood that “biocatalyst” is inclusive of both natural and engineered proteins and small molecules, as well as proteins that have been chemically synthesized. “Biocatalyst” as used herein is intended to include small molecules of the type described in an article titled “Bifunctional Small Molecules Are Biomimetic Catalysts for Silica Synthesis at Neutral pH”, by K. M. Roth, Y. Zhou, W. Yang, and D. E. Morse, J. Am. Chem. Soc., Vol. 127, pp. 325-330 (2005). “Proteins” as used herein is inclusive of polypeptides and oligopeptides, i.e., polymers containing 10 or more amino acids or amino acid analogs, as well as amino acids that have biologically or chemically modified after incorporation into the protein.

Exemplary biocatalysts also include certain long-chain polyamines, for example, poly(L-lysine) (PLL) and poly(ethyleneimine) (PEI), which can catalyze the deposition of minerals from mineral precursors. PEI can be derived, for example, from the polymerization of ethyleneimine, yielding a highly branched hydrophilic three-dimensional matrix. In one embodiment, about 25% of the resultant amines are primary, about 50% are secondary, and about 25% of the amines are tertiary. Poly(glutamic acid) (PGA) can also be used, which can catalyze the formation of calcium phosphates such as hydroxylapatite. Other biocatalysts suitable for promoting the deposition of silica occur naturally in primitive marine species such as the sponge Tethya aurantia and the diatom Cylindrotheca fusiformis. About 75% of the dry weight of the sponge T. auranti is constituted by silica spicules. Each spicule contains a central protein filament, shown by x-ray diffraction to exhibit a highly regular, repeating structure. The nucleic acid sequences for silicatein alpha and beta provided as SEQ ID NO. 1 and SEQ ID NO. 2, respectively. The protein filaments can be dissociated to yield three similar subunits, named silicatein alpha, beta, and gamma. The molecular weights and amino acid compositions of the three silicateins are similar, suggesting that they are members of a single protein family. The amino acid sequences for silicatein alpha, beta, and gamma are provided as SEQ ID NO. 3, and SEQ ID NO. 4, respectively. The cDNA sequence of silicatein alpha, the most abundant of these subunits, reveals that this protein is highly similar to members of the cathepsin L and papain family of proteases. The cysteine at the active site in the proteases is replaced by serine in silicatein alpha, although the six cysteines that form disulfide bridges in the proteases are conserved. Silicatein alpha also contains unique tandem arrays of multiple hydroxyl groups. These structural features can help explain the mechanism of biosilicification and the recently discovered activity of the silicateins in promoting the condensation of silica and organically modified siloxane polymers (silicones) from the corresponding silicon alkoxides, such as tetraethyl orthosilicate (TEOS).

Diatoms also contain proteins of long-chain polyamines and polycationic polypeptides termed silaffins, which catalyze the deposition of silica. Two silica-precipitating peptides, silaffin-1A₁ and -1A₂, both encoded by the sill gene (SEQ ID NO. 5) from the diatom C. fusiformis, have been extracted from cell walls and purified to homogeneity. Silaffin-1A₁ (SSKKSGSY SGSKGSK; SEQ ID NO. 6) and -1A₂ (SSKKSGSYSG YSTKKSGS; SEQ ID NO. 7) consist of 15 and 18 amino acid residues, respectively. Each peptide contains a total of four lysine residues, which are all post-translationally modified. In silaffin-1A₂ the lysine residues are clustered in two pairs in which the ε-amino group of the first residue is linked to a linear polyamine consisting of 5 to 11 N-methylated propylamine units, whereas the second lysine is converted to ε-N,N-dimethyllysine. Silaffin-1A₁ contains only a single lysine pair exhibiting the same structural features. One of the two remaining lysine residues was identified as ε-N,N,N-trimethyl-δ-hydroxylysine, a lysine derivative containing a quaternary ammonium group. The fourth lysine residue again is linked to a long-chain polyamine. Silaffin-1A₁ is the first peptide shown to contain ε-N,N,N-trimethyl-5-hydroxylysine. In vitro, both peptides precipitate silica nanospheres within seconds when added to a monosilicic acid solution.

Silaffin R5 is a 19 amino acid section of the full natural protein, having the sequence SSK KSG SYS GSK GSK RRIL (SEQ ID NO. 8). This sequence is one of the repeating units homologous to the 265 amino acid polymer of the diatom C. fusiformis.

Amelogenins, which control the growth of enamel (a form of hydroxylapatite), are high molecular weight proteins that self-assemble to form nanospheres that, in turn, direct the precipitation of hydroxylapatite in the form of elongated needles. The proteins consist of five domains of amino-acid sequences: (i) protein-protein interacting domain, (ii) lectin-like binding tri-tyrosine domain, (iii) hydrophobic domain (bulk of protein), rich in Pro, Hys, and Gln residues, (iv) an enamelysin-cleavable domain, and (v) mineral-binding hydrophilic domain. Such proteins are described, for example, by J. Moradian-Oldak, in Matrix Biology, Vol. 20, (2001), pp. 293-305.

Bone sialoprotein (BSP) is a sulphated and phosphorylated sialoglyco-protein that regulates the formation of hydroxylapatite crystals during bone formation. The protein is rich is glutamic acid residues. BSP has been described, for example, by Coralee E. Tye, Graeme K. Hunter, and Harvey A. Goldberg, in THE JOURNAL OF BIOLOGICAL CHEMISTRY, Vol. 280, No. 14, Issue of April 8, pp. 13487-13492 (2005).

The biocatalysts can be isolated from the biological organisms by methods known in the art. For example the biocatalyst can be prepared by extraction from organism's tissues containing the biocatalyst. Alternatively, the biocatalyst can also be produced from a recombinant organism into which a cloned gene of the biocatalyst has been introduced. When produced in this manner, the biocatalyst can be linked with certain promoters as is known in the art, to increase production of the catalyst. It is also possible to link the biocatalyst with other proteins that enhance or otherwise modify mineral deposition. For example, native silaffin-2 (natSil-2) is a highly polyanionic phospho-protein that carries unconventional amino acid modifications. NatSil-2 lacked intrinsic silica formation activity but was able to regulate the activities of the previously characterized silica-forming biomolecules natSil-1A. Work by Poulsen et al. has shown that combining natSil-2 (SEQ ID NO. 9) and natSil-1A (SEQ ID NO. 10) (or long-chain polyamines) generated an organic matrix that mediated precipitation of porous silica within minutes after the addition of silicic acid. The precipitate displayed pore sizes in the range 100-1000 nm, which is characteristic for diatom biosilica nanopatterns.

The biocatalysts that promote the deposition of a mineral from a mineral precursor can be an enzyme, an analog of an enzyme, an enzyme fragment, an analog of an enzyme fragment, or a modified protein derived from, for example, DNA shuffling of an enzyme, so long as the analog, fragment, or modified protein is effective in promoting the deposition of a mineral from a mineral precursor.

Analogs include a substantially homologous polypeptide encoded by the same genetic locus in an organism, i.e., an allelic variant, as well as other splicing analogs. Analogs also encompass polypeptides derived from other genetic loci in an organism, but having significant homology to a polypeptide encoded by the biocatalytic gene. Analogs also include proteins substantially homologous or identical to these proteins but derived from another organism, i.e., an ortholog. Analogs also include proteins that are substantially homologous or identical to these proteins that are produced by chemical synthesis. Analogs also include proteins that are substantially homologous or identical to these proteins and that are produced by recombinant methods. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990). An analog polypeptide can differ in amino acid sequence by one or more substitutions, deletions, insertions, inversions, fusions, and truncations or a combination of any of these. Further, analog polypeptides can be fully functional or can lack function in one or more activities. Fully functional analogs typically contain only conservative variation or variation in non-critical residues or in non-critical regions. Functional analogs can also contain substitution of similar amino acids that result in no change or an insignificant change in function. Alternatively, such substitutions may positively or negatively affect function to some degree. Non-functional analogs typically contain one or more non-conservative amino acid substitutions, deletions, insertions, inversions, or truncation or a substitution, insertion, inversion, or deletion in a critical residue or critical region. Amino acids that are essential for function can be identified by methods known in the art, such as site-directed mutagenesis or alanine-scanning mutagenesis (Cunningham et al., Science, 244:1081-1085 (1989)). Sites that are critical for polypeptide activity can also be determined by structural analysis such as crystallization, nuclear magnetic resonance or photoaffinity labeling (Smith et al., J. Mol. Biol., 224:899-904 (1992); de Vos et al. Science, 255:306-312 (1992)).

“Substantially homologous” as used herein in connection with an amino acid or a nucleic acid sequence includes those sequences having a sequence homology or identity of approximately 60% or more, e.g. 70%, 80%, 90%, 95%, 98% or more with a particular sequence and also functionally equivalent variants and related sequences modified by single or multiple base or amino acid substitution, addition and/or deletion. By “functionally equivalent” in this sense is meant nucleotide sequences that encode functionally active biocatalysts that have the ability to promote the deposition of a mineral from a mineral precursor, or amino acid sequences comprising such functionally active peptides. Such functionally equivalent variants may include synthetic or modified amino acid or nucleotide residues provided that the function of the molecule as a whole is retained.

Homology may be assessed by any convenient method. However, for determining the degree of homology between sequences, computer programs that make multiple alignments of sequences are useful, for instance Clustal W (Thompson, J. D., D. G. Higgins, et al. (1994). “CLUSTAL W: Improving the sensitivity of progressive multiple sequence alignment through sequence weighting, position-specific gap penalties and weight matrix choice”. Nucleic Acids Res. 22: 4673-4680). Programs that compare and align pairs of sequences, like ALIGN (E. Myers and W. Miller, “Optical Alignments in Linear Space”, CABIOS (1988) 4: 11-17), FASTA (W. R. Pearson and D. J. Lipman (1988), “Improved tools for biological sequence analysis”, PNAS 85:2444-2448, and W. R. Pearson (1990) “Rapid and sensitive sequence comparison with FASTP and FASTA” Methods in Enzymology 183:63-98) and gapped BLAST (Altschul, S. F., T. L. Madden, et al. (1997). “Gapped BLAST and PSI-BLAST: a new generation of protein database search programs”. Nucleic Acids Res. 25: 3389-3402) are also useful for this purpose. Furthermore, the Dali server at the European Bioinformatics institute offers structure-based alignments of protein sequences (Holm, J. of Mol. Biology, 1993, Vol. 233: 123-38; Holm, Trends in Biochemical Sciences, 1995, Vol 20: 478-480; Holm, Nucleic Acid Res., 1998, Vol. 26: 316-9).

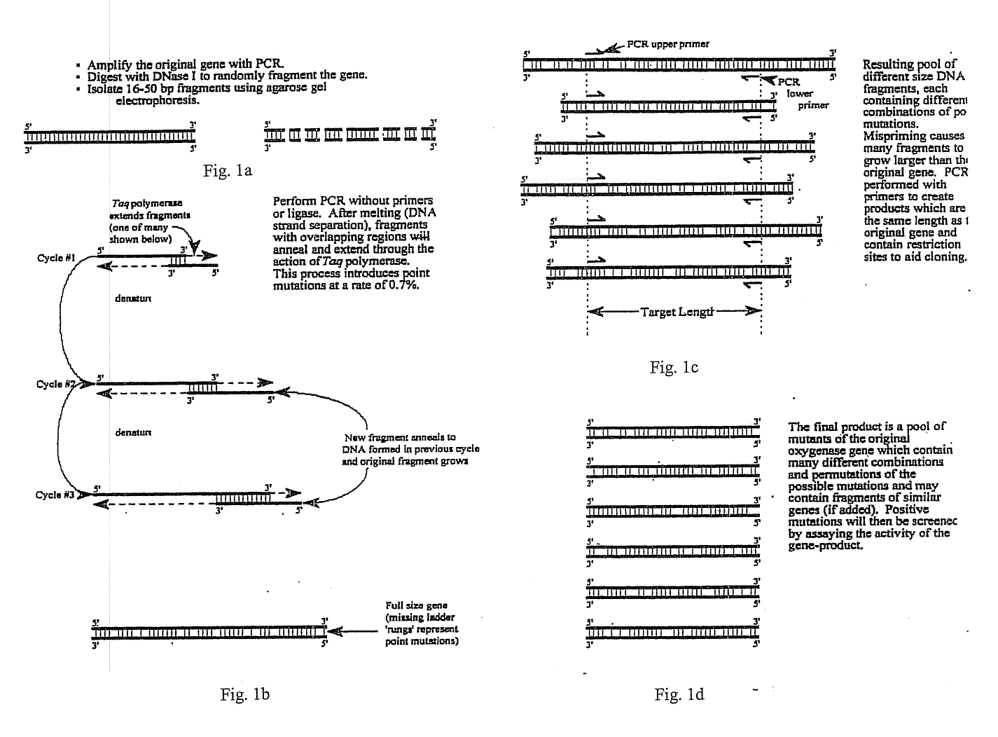

Any biocatalyst having an altered action that is created by modifying the wild-type biocatalyst gene can be used, as long as it retains mineral-depositing activity. In a particular embodiment, the biocatalyst is genetically engineered in order to alter its natural activity or other properties, for example, stability. “DNA shuffling,” also known as “directed evolution” methodology can be used to generate, identify, and isolate highly active biocatalysts selected through screen-specific tests for the formation of different morphologies (shape, size, connectivity, density) of the same mineral; mineralization rate; mineralization using different minerals from different precursors; mineralization activity levels that vary with environment (for example, pH); biocatalyst stability under different conditions, for example, solvent, polymer type, pH, temperature; and the like. This method was developed by Willem Stemmer of Affymax Research Institute (now Maxygen, Inc.) and is shown schematically in FIG. 1.

The method consists of using the polymerase chain reaction (PCR) without oligo primers to re-assemble a gene from random 10-300 bp DNA fragments generated by first cleaving the gene with Dnase. After re-assembling the original gene from these 300 bp fragments using a series of homologous recombinations and extensions with dNTPs and polymerase, normal PCR (with nested oligos) is performed using traditional oligos to yield the full-length gene with random mutations. The mutations arise from infidelity in the assembly process, PCR infidelity (polymerase base reading errors), and errors introduced in the assembly process by insertion of mutated gene fragments (controlled by the researcher by adding specific oligos or DNA fragments from related but not identical genes). The advantages of this method are that DNA shuffling introduces mutations much more efficiently than other methods (for example, unlike DNA shuffling, error-prone PCR and oligonucleotide cassette mutagenesis are not combinatorial), and it can be used to create a chimeric gene by reassembling closely related genes (molecular breeding). This method can be used here to increase the activity of any enzyme that catalyzes mineral deposition, for example both R5 (19 amino acids) and silicatein (208 amino acids) for silica formation. DNA shuffling is performed using this procedure with up to six or more genes, allows, after one round, the generation of up to 20,000 mutants or more. Mutants are screened using an assay as described below. The best mutants identified in the screen are then further assayed, for example to determine yield over a range of precursor concentrations.

Molecular breeding (combining similar genes from different bacteria to introduce even greater genetic diversity) can be conducted in the same manner using the various genes that have been discovered to date for biocatalysts that promote mineralization. This method introduces even greater genetic diversity and can lead to even larger increases in enzymatic activity for mineralization. The study of sequence changes in the evolved peptides can also aid understanding of the relationship between the mutants and their biocatalytic activity. Such understanding can lead to design of biocatalysts having specific activities.

The biocatalyst is then combined or immobilized with a polymeric matrix material to form a biocatalyst-polymer composite. In one embodiment, the immobilized biocatalyst is localized at the surface of the polymeric matrix material in order to facilitate exposure to the mineral precursor. Alternatively, or in addition, the biocatalyst is coated onto and/or immobilized (incorporated) directly into the polymer matrix material. A variety of methods can be used, including coating, mixing, impregnation, and the like. The biocatalyst can be in solution, suspension, dispersion, or other forms when it is contacted with the polymeric matrix material. In a specific embodiment, the biocatalyst can be combined with a polymeric matrix precursor composition, followed by conversion of the precursor composition to the polymer matrix material, for example by curing or crosslinking.

Useful polymeric matrix materials that are suitable for use in dental applications, do not significantly adversely affect the activity of the biocatalyst, and allow mineralization when the biocatalyst is exposed to a mineral precursor. The materials also have sufficient strength, hydrolytic stability, and non-toxicity to render them suitable for use in the oral environment. Such matrix materials are generally formed from thermosetting (hardenable) materials capable of being hardened to form a polymer network including, for example, acrylate-functional materials, methacrylate-functional materials, epoxy-functional materials, vinyl-functional materials, carbamoylisocyanurate-functional materials, vinyl ether-functional materials, oxetane-functional materials, spiro-orthocarbonate-functional materials, spiro-orthoester-functional materials, and mixtures comprising at least one of the foregoing. The hardenable material can be in the form of a matrix-forming oligomer, monomer, polymer, or blend thereof.

One class of preferred hardenable materials includes materials having free radically active functional groups, or cationically active functional groups. In another alternative, a mixture of hardenable resins that include both cationically curable and free radically curable materials can be used for the dental materials. In still another alternative, the hardenable resin can be a material that includes both cationically active and free radically active functional groups in the same molecule.

Suitable materials having free radically active functional groups contain at least one ethylenically unsaturated functional group, and are capable of undergoing addition polymerization. Such free radically polymerizable materials include mono-, di- or poly-acrylates and methacrylates such as methyl acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexaacrylate, the diglycidyl methacrylate of bis-phenol A (“Bis-GMA”), bis[1-(2-acryloxy)]-p-ethoxyphenyldimethylmethane, bis[1-(3-acryloxy-2-hydroxy)]-p-propoxyphenyldimethylmethane, and trishydroxyethyl-isocyanurate trimethacrylate; the bis-acrylates and bis-methacrylates of polyethylene glycols of molecular weight 200-500; 2-hydroxy ethyl methacrylate (HEMA) and vinyl compounds such as styrene, diallyl phthalate, divinyl succinate, divinyl adipate and divinylphthalate. Mixtures of at least one of these free radically polymerizable materials can be used if desired.

For free radical polymerization (for example, hardening), an initiation system can be selected from systems that initiate polymerization via radiation, heat, or redox/auto-cure chemical reaction. Suitable free radical-generating photoinitiators generate free radicals for addition polymerization upon exposure to light energy having a wavelength between 200 and 800 nm, and are optionally combined with a photosensitizer or accelerator. Exemplary systems include an amine and an α-diketone; a three component system such as an iodonium salt (for example, a diaryliodonium salt), a sensitizer, and a donor; and an acylphosphine oxides and a tertiary amine reducing agents. Heat can be used to initiate the polymerization of free radically active groups, optionally in the presence of radical-generating thermal initiators such as peroxides (for example, benzoyl peroxide and lauryl peroxide) and azo compounds (for example, 2,2-azobisisobutyronitrile (AIBN)). Suitable heat sources should be capable of generating temperatures of about 40° C. to about 150° C. under normal conditions or at elevated pressure, and are generally preferred for initiating polymerization of materials outside of the oral environment. Chemical initiator (auto-cure) systems are exemplified by a combination of a peroxide and an amine, and are typically supplied as two-part systems in which the reactants are stored apart from each other and then combined immediately prior to use.

Materials having cationically active functional groups, such as cationically polymerizable epoxies, vinyl ethers, oxetanes, spiro-orthocarbonates, spiro-orthoesters, and the like can also be used. Optionally, monohydroxy and polyhydroxy alcohols can be added as chain-extenders for these materials, particularly epoxy-functional materials. Initiation system can be selected from systems that initiate polymerization via radiation (using, for example, an aryliodonium salt, a sensitizer, and an electron donor; or organometallic complex cations essentially free of metal hydride or metal allyl functionality), heat (using initiators such as anhydrides and/or amines, for example cis-1,2-cyclohexanedicarboxylic anhydride), or redox/auto-cure chemical reactions.

Polymerization generally occurs after placement at the site to be treated, and either before, during, or after mineralization. Where cure is accomplished using chemical- or radiation-induced free radical mechanisms, it is generally preferred to cure after mineralization. Alternatively, polymerization of the polymeric matrix material can also occur simultaneously with the formation of the mineral.

In another embodiment, a swellable polymer matrix material is used. The biocatalyst can be incorporated into such a material by suspending it in a solution, then contacting the solution with the swellable polymer matrix. This technique is effective with a copolymer system formed from 45:45:10 weight % (wt %) n-butyl acrylate, 2-hydroxyethyl acrylate (BA-HEA), and the crosslinker triethylene glycol dimethacrylate, respectively. After polymerization by exposure to UV radiation, this composition yields a durable polymeric solid that absorbs a limited amount of water, specifically 10 wt % water at equilibrium.

The polymer matrix material can further comprise other additives known in the art (for example, particulate fillers, curing agents, UV stabilizers, antioxidants, and the like), or the additives can be incorporated after the biocatalyst.

The biocatalysts can be immobilized prior to incorporation into a polymeric matrix suitable for use as a dental material. The biocatalysts can be immobilized within one material (for example, a carrier), which is then incorporated into the polymeric matrix material. Alternatively, the biocatalyst can be immobilized directly into the polymer matrix material. Immobilization can occur by the formation of non-covalent bonds or linkages, such as those associated with ionic or electrostatic interactions, or with van der Waals forces, or immobilization can occur through the formation of covalent bonds between the catalyst and the carrier or polymer matrix material. Physical protein-immobilizing or capturing vehicles, such as enzyme-entrapment or encapsulation can also be used.

Materials and techniques for the immobilization of biocatalysts are known, and selected so as to effectively immobilize the biocatalyst while allowing sufficient mineral-depositing activity; stabilize the biocatalyst if desired; and provide permeability to the mineral precursor. In one embodiment, the immobilization material preferably stabilizes the biocatalyst so that the biocatalyst retains its activity for about 30 days to about 365 days. The retention of activity is defined by the number of days that the biocatalyst retains at least about 75% of its initial activity. Specific examples include, but are not limited to, inorganic carriers such as porous ceramic particles, porous silica gel particles and zeolite particles; natural polymer gels such as agar, calcium alginate and chitosan; and synthetic polymer gels such as polyacrylic acid, polyacrylamide and polyvinyl alcohol. The biocatalyst can be immobilized, for example, by allowing carriers to absorb a biocatalyst solution, by mixing carriers with a biocatalyst solution to immobilize enzyme molecules on/in the carriers by absorption, by entrapping and immobilizing enzyme molecules within carriers, or by cross-linking enzyme molecules via crosslinkers.

An additional feature of immobilization is the ability of the immobilization material to act as a template for mineralization by imparting the pattern of its structure on the deposited mineral. For example, where the biocatalyst is immobilized on fibers or multilayer coatings, the resulting mineral deposits can be in fibrous or layer form. This feature offers structure (and thus property) control for specific application requirements, and will be described in more detail below. In general, control of morphology can be important whether it is due to the immobilizing material or other aspects of the catalyzed mineralization reaction.

As described above, the biocatalyst is deposited onto the polymer matrix and adsorbed and/or absorbed onto the polymer surface. It is also possible to incorporate the biocatalyst within certain polymer matrices to provide a polymer/biocatalyst composite. These composites can then have further biocatalyst adsorbed and/or absorbed onto the composite surface.

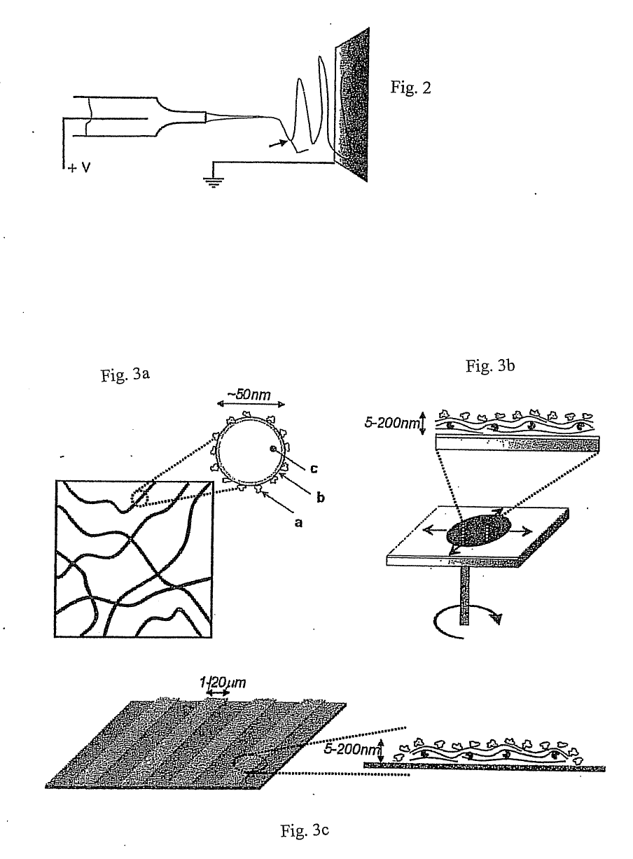

Thus, in one embodiment, the mineralization biocatalyst is incorporated into nanoscale diameter electrospun fibers, which are described below in connection with FIG. 2. The high surface-to-volume ratio of these fibers allows increased silica deposition, and the fibers are readily incorporated into the polymeric matrix material. Suitable fibers can have average diameters of about 1 to about 100 nanometers (nm), specifically about 10 to about 90 nm. The fibers can also have average diameters of about 10 to about 20 nm, about 20 to about 30 nm, about 30 to about 40 nm, about 40 to about 50 nm, about 50 to about 60 nm, about 60 to about 70 nm, about 70 to about 80=n, about 80 to about 90 nm, or about 90 to about 100 nm. The aspect ratio (length:diameter) of the nanofibers is selected based on factors such as availability and ease of incorporation into the polymeric matrix material. Electrospun nanofibers can be characterized by their “persistence length” ratio (Lₚ/D). The persistence length, Lₚ, is the length over which the fiber is locally straight, that is, deviating from a locally defined tangent by less than 10%. The electrospun nanofibers can have an Lₚ/D ratio of about 2 to about 1000.

The fibers can be spun from a variety of polymers. The polymers are preferably compatible with the enzyme and the polymeric matrix, and are also approved for use in the human body, i.e., nontoxic. The polymers also maintain mechanical integrity in saliva, or other pertinent body fluid, by not dissolving or degrading over the timescale required for the desired degree of biocatalyst activity. Exemplary polymers include poly(vinyl alcohol) (PVA) and copolymers of vinyl alcohol, for example, poly(ethylene-co-vinyl alcohol), crosslinked poly(ethylene oxide), and the like.

In another embodiment, stratified multilayers of biocatalyst are formed, using a process known as layer-by-layer (LbL) assembly. The LbL procedure affords the opportunity to more precisely position the biocatalyst within the thickness of the polymer matrix. LbL is essentially a process of alternately applying polymers of opposite charge onto a substrate. By controlling the dipping, casting, or spinning process the thickness of the polyelectrolyte multilayers can be controlled. LbL assembly can be used in actual clinical applications, i.e. chairside where a limited number of layers (for example 1-5) are deposited. LbL assembly can also be useful in the dental laboratory setting to provide forms or scaffolds that are pre-assembled and then used chairside.

LbL assembly has been described in the art, and has become a nanoscale processing tool applicable to many polymeric and colloidal materials. In this method, a charged substrate is exposed to dilute solutions of charged macromolecules (polyelectrolytes, enzymes, etc.) or colloidal particles so that the long-ranged electrostatic attraction affords accelerated adsorption with overcharging. Thus, sequential exposure of a substrate to alternating solutions of polyanions and polycations allows facile build-up of highly interpenetrated multilayers with significant expulsion of counterions and thickness resolution of 2-5 nm per bilayer pair. In the past, this method has been implemented mostly in a “dip-coating” configuration; however, there has been a significant drive toward extending the process to include more rapid fabrication and more versatile structures. Two procedures have been developed to meet this need, (i) spin self-assembly (SSA), which uses a traditional spin-coating configuration, with sequentially applying dilute polyelectrolyte solutions of opposite charge, and (ii) flow self-assembly (FSA), localized multilayer deposition in microfluidic channels. In the latter method, described in more detail below, solutions of polyanions and polycations are alternately flowed through narrow channels defined by a microfluidic network (μFN). The μFN has been prepared, for example, with soft lithography. Upon alternate flow of the polyanions/polycations, nanometer-resolved coating is deposited on a substrate with micrometer-scale localization designed at the photolithography stage of μFN processing.

In the FSA process, which couples μFN flow and LbL adsorption, spatially patterned multilayers are formed by using a microfluidic “mask” that promotes the formation of two-dimensional multilayer arrays with dimensions matching the μFN microchannels. These dimensions are about 10 to about 30 μm in width. By using microfluidics devices, the deposition of polyelectrolytes onto the surface can be localized, such that only the uncovered parts of the substrates are available to deposition whereas, the covered regions will remain unchanged. Thus multilayer deposition is guided with microfluidic networks as conduits for patterned delivery of chemical reactants onto a substrate.

In this approach, the microfluidics networks (μFNs) are made of a highly compliant and conformably sealing elastomer, such as crosslinked polydimethylsiloxane (PDMS). When applied to a substrate, the structured elastomer seals the surface by its conformal contact and makes linked, closed capillaries, which further guide any filling material for deposition onto the substrate surface with great fidelity to the pattern imprinted in the elastomer. Deposition is expected to occur where the channels are located at the surface of the substrate and when using adsorbing macromolecules such as polyelectrolytes. PDMS is a good stamp material for this method because of its rubber elasticity and ease of handling, although any elastomer with softening temperature (T_{g} or Tₘ) below room temperature can function well.

Various embodiments of the above templating approaches are described in reference to FIG. 3, which shows three geometries of nanometer-scaled scaffolding. In case (i) polymeric nanofibers are electrospun from aqueous solution with the biocatalyst present in the interior or on the exterior, so that exposure to a mineralization reagent is catalyzed for rapid and tethered precipitation. In case (ii), a 1D (z-axis) nanostratified polymeric scaffold prepared by SSA as described above is shown. In case (iii), polymeric multilayers having dimensions of 1-20 μm in width is shown. Other geometries and configurations, for example helices, are also within the scope of the invention.

In one specific embodiment, biocatalyst/polymer multilayer films have been prepared on silicon wafers using an LbL process. After etching of the silicon wafer to produce a hydroxylated surface, a first layer, e.g., polyethyleneimine (PEI) is deposited by dipping the substrate for a sufficient time, for example, 15 minutes. Subsequently, multilayers are formed by alternating deposition of a second layer, e.g., poly(L-lysine) (PLL) followed by poly(styrene sulfonate) (PSS). The LbL process can be repeated until multilayer films, for example, PEI-(PLL/PSS)ₘ, PEI-(PEI/PSS)ₙ, or PEI-(PEI/PSS/PLL/PSS)ₚ, wherein m, n, and p are independently integers of 2 to 100, more specifically, 2 to 50, or more specifically, 2 to 20, or even more specifically, 2 to 10, are formed. As used herein, the subscript m, n, or p means the number of alternating layers. For example, multilayer film PEI-(PLL/PSS)ₘ means a first layer of PEI followed by m alternating layers PLL and PSS. In one embodiment, the catalytic multilayer film is a PEI-(PLL/PSS)₁₀ film.

Mineral precursor solutions, for example, pre-hydrolyzed alkoxysilane solutions, are placed on the catalytic multilayer films described above for mineralization. Several different factors, for example, precursor concentration, solvent, and drying conditions, can have an effect on the morphology, roughness, and contact mechanical stiffness of the formed mineral. In one embodiment, the morphology of the silica formed on the catalytic multilayer films is plate-like or spherical, and porous with average particle size depending on the catalyst and its position on the surface. Without a catalyst the silica formed over longer times has a fine, gel-like appearance. The morphology of silica produced on the multilayer films is different from that of particles catalyzed in solution with the same biocatalyst.

It has further been found that the homogeneity of the PEI-(PLL/PSS)₁₀ films increases with increasing drying temperature, silica precursor concentration, and the presence of ethanol. The contact mechanical stiffness of the silica particles (40 N/m) catalyzed from PEI-(PLL/PSS)₁₀ films is lower than the non-silicified areas (48 N/m), suggesting that regions of the silica are amorphous and hydrated. These results show that a biocatalyst applied to a surface as a multiple layer with an oppositely charged polymer host (PSS) maintains its activity for silicification. The generally coherent nature of the mineral coating suggests its potential for enhancing critical restorative dental interfaces.

The biocatalyst is combined with the polymeric matrix material in an amount sufficient to provide an effective degree of mineralization, for example, silicification of the polymeric matrix. The effective degree of mineralization will vary depending on the particular use of the material. For example, a part of a restoration that will come into chewing contact with other teeth can require a higher degree of mineralization than a part that does not. In general, an amount of biocatalyst is combined that provides greater than about 50 wt %, more specifically, greater than about 65 wt %, or more specifically, greater than about 80 wt %, or even more specifically, greater than about 90 wt % mineral, based on the total weight of the total material (polymer matrix and mineral).

After incorporation of the biocatalyst, the biocatalyst-polymer composite is exposed to a mineral precursor composition. The precursor is preferably stable in an aqueous environment, since many biocatalysts function in water. When the mineral is a ceramic such as silica, suitable precursors can be tetramethoxy silane (TMOS), tetraethoxy silane (TEOS), or silicic acid (Si(OH)₄). One precursor that is converted to silica by R5 or other biocatalysts is tetraethoxy silane (TEOS, Si(OCH₂CH₃)₄). The TEOS is dissolved in an aqueous solution at a pH that is compatible with the function of the biocatalyst, for example slightly acidic. Silicification occurs upon contact of the biocatalyst with a silica precursor. In an advantageous feature, the biocatalyst enables mineral formation to occur within clinically viable conditions of temperature (for example, 20-50° C.), time (for example, 1 minute to several hours), and pH (for example, about pH 7), unlike the uncatalyzed reactions that require low or high pH and elevated temperatures.

In one embodiment, the mineral is deposited in the polymer matrix material prior to placement in a restoration or site to be restored. This produces a biocatalyst-polymer-mineral composite that can be used in the same manner as other dental ceramic-polymer composite materials.

In another embodiment, the biocatalyst-polymer composite or the biocatalyst-polymer-mineral composite is applied to a restoration or site to be treated, and then exposed to a mineral precursor. For example, contacting the biocatalyst-polymer composite with a precursor solution such as a TEOS solution initiates diffusion of the precursor into the polymeric host as driven by a strong chemical potential gradient, but mediated by condensation polymerization biocatalyzed by the immobilized R5 polypeptide or variant. As polymerization ensues, further diffusion of mineral precursor into the polymeric host is retarded until a final, graded, state is achieved. This graded structure is anticipated to have enhanced durability compared to a coating of the same composition.

The depth of the gradient will depend on the amount of the biocatalyst that was incorporated into the bulk polymer matrix material, the rate of deposition, the rate of diffusion, and other considerations. Increased rates of deposition will result in less penetration of mineral precursor, and thus thinner gradients, while increased concentrations of mineral precursor will result in increased diffusion and thus thicker gradients. Thus, the thickness of the gradient can be adjusted by adjusting the biocatalytic activity and mineral precursor concentration. In one embodiment the mineral gradient spans about 0.1 to about 3.0 millimeters (mm), more specifically about 0.5 to about 1.5 mm.

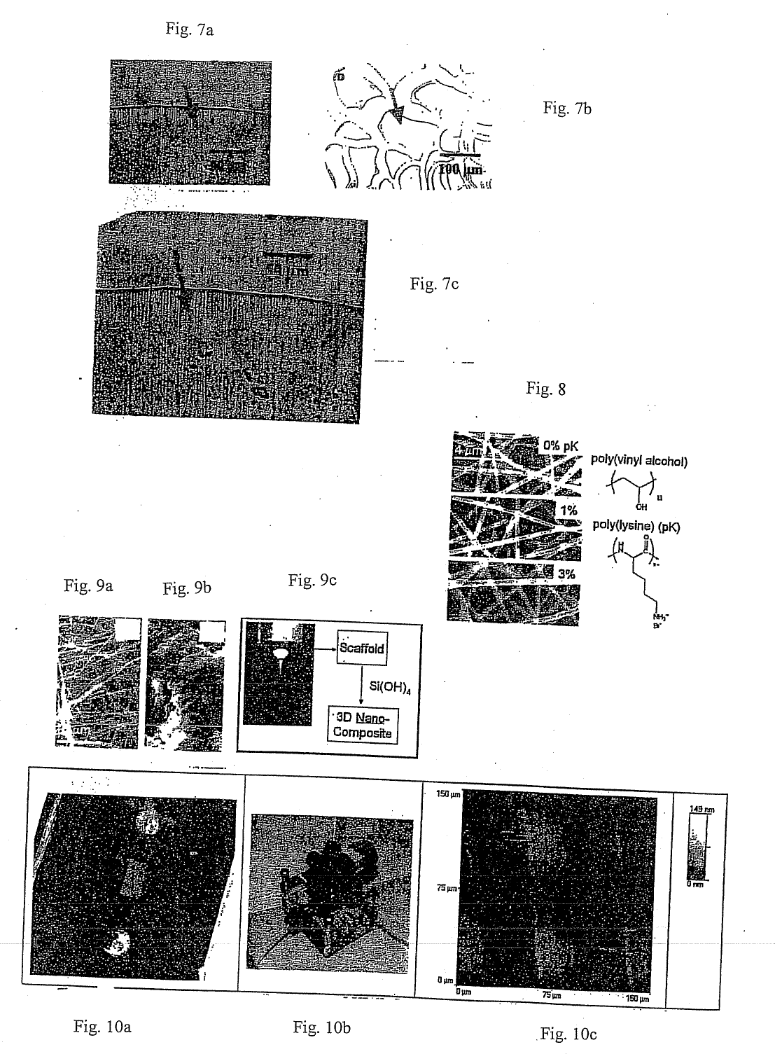

While Presently Available Biocatalysts Often Result in the Deposition of a Mineral of a specific form, alteration of the biocatalyst structure can alter the form of the deposited mineral. For example, silica precipitated by R5 can be amorphous. However, it has been shown that slight mutations can alter the morphology/topography of both the primary particles and their aggregates. The silica precipitated in Example 2 had distinct striations (FIGS. 7a and 7c) when viewed with optical microscopy, but further characterization is needed to determine crystallinity. The natural biocatalyst discovered by Cho, Morse and others was filamentary and formed ceramic needles. It can therefore be the case that R5 or other small proteins themselves can be used as templates to form supramolecular structures.

For example, the biocatalaysts could be formed into nanoscale fibers using electrospinning. The biocatalytic nanofibers would catalyze mineral directly using the fiber as a template. Electrostatic spinning of polymeric fibers has been shown previously to allow the manufacture of fibers from commodity-type polymers with diameters as small as 10 nm. In the electrostatic spraying of liquids, the liquid to be sprayed is charged to a high potential relative to an electrically grounded surface nearby. At a high electric potential (>+10 kV), significant positive charge builds up on the surface closest to the electrical ground, causing a “Taylor cone” to be formed and, subsequently, a charged jet to be accelerated toward the ground. When applied to ordinary liquids, this jet quickly fragments into droplets that continue to accelerate to the ground, where they ultimately form a coating. When the same apparatus is applied to polymeric liquids, either solutions of polymer in a solvent or molten polymers without solvent, fragmentation of the ejected filament is greatly suppressed and long, semi-continuous filaments result. The ejected filaments are then subject to a “whipping” convective instability wherein nonaxisymmetric fiber distortions grow exponentially, allowing for large total fiber draw ratios and thus small diameters approaching 10 nm. The general processing schematic is shown in FIG. 2. On the left is a delivery nozzle containing liquid that is charged to a high electric potential with an immersed electrode. A conducting wall of the tube can also serve as the electrode. The grounded screen to the right collects the nanofibers. Most research on electrostatic spinning of polymeric fibers to date has focused primarily on isotropic polymer solutions of such common polymers as poly(ethylene oxide), polyurethane, and poly(ethylene terephthalate). Recent results have shown that fibrinogen, type II collagen, and poly(ethylene vinyl alcohol) can also be processed by electrostatic spinning.

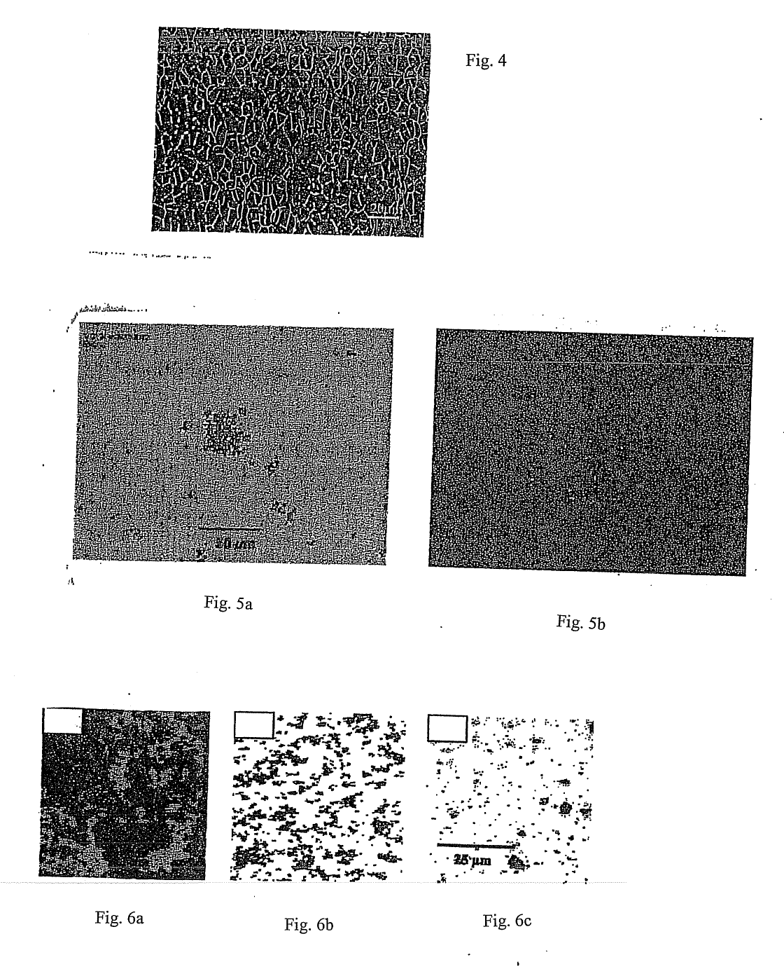

In Another Embodiment, the Biocatalyst is Formed into a Network by Evaporating a polymer solution containing the biocatalyst with forced flow of moist air across the solution surface. Such a process can cause condensation of water droplets on the cool surface of the evaporating polymer solution that, upon sinking into the lower density solution and continued evaporation to dryness, lead to a porous film. The resulting network structure formed from this procedure using PLA (poly(lactic acid)) in a toluene solvent (1 wt % of PLA) is shown in FIG. 4. Empty cells are formed by the approximately 1 μm thick polymer walls. Mineral deposition could proceed by nucleation at the biocatalyst-containing walls, growing to a size and arrangement dictated by the architecture of the network.

The biocatalyst-polymer composite can be useful in a variety of dental applications, for example restorative materials such as wear-resistant filling materials and pit and fissure sealants, dental bonding agents, dental sealants, restoration of root caries, desensitizing agents, dental materials or coatings containing therapeutic agents, agents for the control of demineralization/remineralization processes, agents for formation of mineral to restore or repair teeth, agents applied to dental tissues that then control mineralization or formation of protective coatings, agents for endodontic procedures, orthodontic wires and brackets with hardened surfaces for control of friction, dental materials that form mineral in response to changes in the environment, coatings for improving bone contact (osseointegration) for dental implants, and surgical agents (oral surgery, periodontology) that form mineral to stimulate bone healing or growth.

Dental restorations using the biocatalyst-polymer composites can be made by a variety of different methods. In one embodiment, a biocatalyst is incorporated into a bulk polymer material, and the bulk polymer material is contacted with a mineral (for example, a silica or a calcium phosphate) precursor. After the desired degree of mineralization has occurred, the bulk polymer is formed into a dental restoration, and the bulk polymer material is optionally cured.

In another embodiment, a bulk polymer material is formed into a dental restoration, and a bulk polymer material containing the biocatalyst is applied onto at least a portion of the surface thereof. The polymer material is then contacted with a mineral precursor until the desired degree of mineralization is attained. The bulk polymer material can be cured before or after mineralization. Alternatively, the bulk polymer material can also be cured simultaneously with mineralization.

In another embodiment, the biocatalyst-polymer composite is used as a bonding agent. In situ formation of mineral could improve the bonding and sealing capability of the polymer matrix, including reduction of microleakage between a restoration and the tooth structure, and the sealing of dentin tubules to minimize fluid flow and sensitivity.

Use of other biocatalysts, in addition to those described above, is also within the scope of the present invention. For example, amelogenin forms supramolecular nanospheres that interact with the growing hydroxylapatite (HA) crystallites and affect crystal size and orientation in the tooth structure. In addition, it has recently been shown that the full dentin matrix protein 1 is essential for mineral nucleation in that a particular conformation of the active areas of the enzyme is critical since apatite crystallization was not achieved by small peptides. Incorporation of one or more of these biocatalaysts could be used to supplement the mineralization provided by the biocatalysts described herein. Other additives, for example therapeutic agents such as antimicrobial, antibacterial or bone growth stimulating agents can also be included.

Restorations prepared by biomimetic catalysts are expected to possess an unusual combination of toughness and hardness through the combination of a bulk polymer with a hybrid polymeric/ceramic surface, much like the structure of dentine and enamel in a tooth. The composites should also have high luster. Other characteristics, such as water absorption, can be adjusted by varying the polymer matrix.

The invention is further illustrated in the following non-limiting examples. In the examples, all purchased reagents were used as received unless otherwise noted and, unless otherwise noted, all procedural steps in the following sections were carried out at room temperature.

### EXAMPLES

### Example 1

### Silicification in Solution Using R5 as Biocatalyst

Silicification involves simultaneous hydrolysis and condensation reactions. The hydrolysis reaction involves hydrolysis of a silicon precursor compound Si—(OR)₄, wherein each occurrence of R can be independently a hydrogen or a C₁-C₁₀ alkyl. In one embodiment, the silicon precursor compound is silicon alkoxide Si(OEt)₄ (tetraethyl orthosilicate or “TEOS”). The hydrolysis produces silanol (Si—OH such as Si(OH₄), silicic acid) and liberates alcohol molecules. The condensation reaction involves the condensation of two silanol substrates to form siloxane bonds (Si—O—Si), which will eventually form the silica network and the release of water molecules. The water molecules can then hydrolyze another alkoxide precursor molecule. Such sol-gel reactions are common to commercial formulations, but require elevated temperatures and acid or base catalysts. In biocatalyzed mineralization, polypeptides or other charged polymers act as biocatalysts and templates for these reactions, directing the structure of the growing mineral under ambient conditions.

A R5 biocatalyst, a peptide having the sequence SSK KSG SYS GSK GSK RRIL, (SEQ ID NO. 8) was synthesized by Alpha Diagnostic International (79% purity) and was stored at 0° C. and thawed to room temperature for at least 0.5 hours (h) prior to use. Buffer solution for this peptide was prepared by vortexing 0.0021 g of the R5 biocatalyst with 280 microliters (μL) of 0.1 M phosphate buffer (pH 7.4). Silica precursor solution was similarly prepared by vortexing 30 μL of TEOS, purchased from Gelest, with 170 μL of 0.001 M HCl. Both solutions were clear, colorless, and homogeneous.

The above mixtures were stored at 40° C. overnight (no gelation of the TEOS/HCl solution was noted), following which the two were combined in a centrifuge tube (still homogeneous at this point) and re-suspended for approximately one minute. The reaction was allowed to proceed for 15 minutes during which time the mixture became more turbid, indicating that particulate had begun to form. After the allotted time had expired, the reaction mixture was centrifuged and a fine white solid was observed. Optical microscopy at magnifications of 20× and 50× was used to verify silica formation. Microscopy was performed using an Olympus BX-50 microscope with a CRI LC-PolScope module and LinkSys imaging software. 20× and 50× achromatic objectives were employed with a CCD camera for total magnifications of 20× and 500×, respectively.

Microscopy verified the presence of small particles in the reaction mixture containing both R5 and TEOS solution. The size of these spheres is estimated to be approximately 700 nm. FIGS. 5a and 5b show images of an aggregate of these spherical particles at 20× (left) and 50× (right) magnification, respectively.

These experiments were repeated with TEOS solution concentrations of 30%, 15%, and 5% by volume. The mixtures rapidly became turbid, and a drop was placed on a cover slip and examined with light microscopy. Small spheres and their agglomerates were again observed to form, with number density proportional to the original TEOS concentration (FIG. 6a: 30%, FIG. 6b: 15%, and FIG. 6c: 5% TEOS). It is apparent that TEOS concentration also influences the overall morphology.

### Example 2

### Generation of New Mineralization Biocatalysts

To initiate the random mutagenesis for enhanced silica synthesis, a combinatorial DNA mutagenesis approach (DNA shuffling) coupled with a high-throughput (spectrophotometric) can be used. Bacterial surface display (expression) would allow screening of a large number of silica-forming enzymes, as the shuffled protein is expressed on the cell surface, becoming available for reaction with the orthosilicic acid. In particular, the gene for the mature 208 amino acid silicatein from the sponge Tethya aurantia could be shuffled initially for enhanced precipitation of silica. The method of surface display would be that of Cho et al. using the ice nucleation protein. Since no cofactors (for example, NADH) are required for the precipitation of silica, surface display would allow use of bacteria while also avoiding cell lysis and protein purification steps during the screen.

The surface display plasmid with the silicatein gene fragment fused to gene for the ice nucleation protein can be purified with Qiagen MIDI prep kits, and the silicatein gene amplified using high-fidelity PCR using Pfu polymerase. DNA shuffling can be performed using the procedure of Stemmer modified by H. Zhao and F. H. Arnold, Nucleic Acids Research. Vol. 25, No. 6, pp. 1307-08 (1997), with DNA errors introduced primarily during the shuffling reaction so that most of the introduced errors are from mismatches during annealing rather than from polymerase error. To isolate template DNA to be shuffled, PCR is performed on 0.5 μg of the silicatein plasmid in a 100 μL reaction containing 10 mM Tris-HCl (pH 8.3), 50 mM KCl, 10% dimethylsulfoxide (DMSO), 2 mM MgCl₂, 200 μM of each dNTP, 5 U Taq polymerase (Promega), and 0.3 μM of each of the primers. Amplification is carried out in the GeneAmp PCR system 2400 (Perkin Elmer, Norwalk, Conn.).

Fragments for shuffling can be created by digesting the cleaned PCR product with Dnase I in a 50 μL reaction containing 3-5 μg DNA, 50 mM Tris-HCl, pH 7.4, 10 mM MnCl₂ and 0.01 units of Dnase I for 20 minutes at 25° C. The fragments of 20-50 bp are purified using Centri-Sep spin columns (Princeton Separations, Adelphia, N.J.). The fragments are reassembled by PCR without primers in a 50 μL reaction containing 10 mM Tris-HCl, pH 8.3, 50 mM KCl, 10% DMSO, 2 mM MgCl₂ 200 μM of each dNTP, 25 μL Centri Sep DNA fragments and 2.5 units Pfu polymerase (Stratagene, La Jolla, Calif.). The 1.3 kb silicatein gene fragment can be recovered by PCR with primers in a 100 μL reaction containing 1-3 μL of reassembled DNA along with a 1:1 ratio of Taq:Pfu polymerases (2.5 U each).

The PCR product generated is then cloned into the original ice nucleation-silicatein fusion expression plasmid. This shuffled silicatein plasmid library is electroporated into E. coli TG1 using a Bio-Rad Gene Pulser (15 V/cm, 25 μF, 200 Ohm). Electroporation of E. coli TG1 with plasmids containing the shuffled silicatein locus generally yields about 100-200 colonies per plate after incubation for 16 hr at 37° C.

The bacteria expressing the mutant silicateins can be screened spectrophotometrically using live cells by detecting the silica product in a 96-well plate assay based on the silica detection method of Naik et al., Progress in Organic Coatings, Vol. 47, 249 (2003). Bacteria containing mutated silicatein proteins are grown in 300 μL of rich medium at 37° C. with shaking in Costar 96-well plates (Corning, Corning, N.Y.). The cells are harvested at mid-log phase by filtering 200 μL of the cell cultures using MultiScreen-GV 96-well filter plates (Millipore, Bedford, Mass.). The collected cells are then washed with 200 μL 50 mM Tris-HCl, pH 7.4 then suspended in 200 μL of the same buffer. Cell suspensions in the filter plates are then be contacted overnight with shaking with orthosilicic acid prepared by dissolving tetramethyl orthosilicate in 1 mM HCl. The silica products (for example 500-700 nm spheres, as seen in the above results with protein R5 from the diatom Cylindrotheca fusiformis) formed from the reaction with the surface-displayed silicatein are captured by a filtering through a Costar 96-well plate (Corning). After washing the unreacted orthosilicic acid, the precipitated silica is detected by dissolving in 1 M NaOH at 95° C. and reacting with molybdic acid. Monomeric Si(OH)₄ and molybdic acid produce a yellow product, which can be detected at 410 nm, allowing the amount of silica produced by the enzyme variants in each to be quantified spectrophotometrically using a 96-well plate reader (Multiscan RC, Labsystems, Helsinki, Finland).

Bacteria from wells with the highest absorbance are saved, re-checked in additional 96-well plates, and the plasmids isolated from the highest-expressing strains used for subsequent rounds of shuffling as well as DNA sequencing. The sequencing should reveal which amino acids are important in the silicateins. By comparing to a crystal structure, important structure/function relationships can be obtained.

The Same Method Utilized for Silicateins (Bacterial Surface Display) would be Used to evolve the silica-forming peptides derived from diatoms known as silaffins. The small, cationic R5 protein (19 amino acids) from the diatom Cylindrotheca fusiformis represents only a small portion of the protein utilized by the marine organism, which contains 265 amino acids (full-length C. fusiformis silaffin protein (SEQ ID NO. 11)). Since the full-length protein contains seven regions with high homology and a repetitive structure, this approach yields more silica precipitation than a method that just concentrates on a single part of the protein such as R5. The sill locus from C. fusiformis (encoding the full-length silaffin protein) is cloned into the ice nucleation surface display vector as described above, and the entire Sil1 protein is shuffled. Bacteria expressing more active silaffins are screened spectrophotometrically using 96-well plates and the calorimetric screen described above. Molecular breeding of the known silaffins can also be utilized to increase the genetic diversity, which should lead to even greater potential for increasing biocatalyst activity.

### Example 3

### Silicification in Solution Using Poly(L-Lysine) (PLL) as Biocatalyst

In this example, studies of silicification reactions in solutions were conducted to evaluate the effect of several reaction parameters such as solvent and catalyst composition on the formation of minerals.

Silica formations from tetramethyl orthosilicate (TMOS) using three different PLL biocatalysts (low, medium, and high molecular weight as described in Example 18) were analyzed by a calorimetric molybdosilicate assay. As direct hydrolysis and condensation from TMOS was not possible with PLL, TMOS was first prehydrolyzed to form silicic acid. Next, the silicic acid from the prehydrolyzed TMOS was exposed to PLL in solution and the UV absorbance as a function of time was measured and is shown in FIG. 18. The measured absorbance reflects the level of unreacted silicic acid in the reaction medium.

As can be seen from FIG. 18, the conversion to silica saturates after about 40 minutes for each PLL biocatalyst studied. This reaction rate is consistent with requirements in a dental clinical setting.

### Example 4

### Silicification in Solution Using Poly(Ethyleneimine) (Pei) as Biocatalyst

In this example, the efficacy of a synthetic polyamine PEI in accomplishing silicification was studied. Addition of a 3 wt % aqueous PEI solution (PEI has a natural pH is 11) to neat TMOS liquid led to near-instantaneous precipitation (10 to 20 seconds) of silica. Silica formation was rapid in all cases (completing in less than 1 to 2 minutes). The rapid silicification was quantified using thermogravimetric analysis (TGA) for a range of PEI concentrations and TMOS:ethanol (v:v) fractions. The experimental conditions and results are shown in FIG. 19.

As can be seen from FIG. 19, the maximum silica yield (char yield at 800° C. with TGA) was achieved with about 5 wt % PEI and a TMOS:ethanol ratio of about 0.7. The yield was enhanced further with PLL addition (data not shown), probably through the PLL's secondary role as a surface-active component during incipient particle formation important to particle size control.

These results also indicate that PEI can directly catalyze the hydrolysis and condensation of alkoxy-silanes, namely, TMOS and TEOS, to form silica. This avoids the prehydrolysis step and offers the potential benefit of simplification and scalability as pre-hydrolyzed alkoxides are not stable and their uses would complicate translation to practice. Additionally, the short direct silicification timescale of 10 to 20 seconds can also provide great benefits.

### Example 5

### Mineralization of Hydroxylapatite (HA) in Solution Using Poly(Glutamic Acid) (PGA) as Biocatalyst

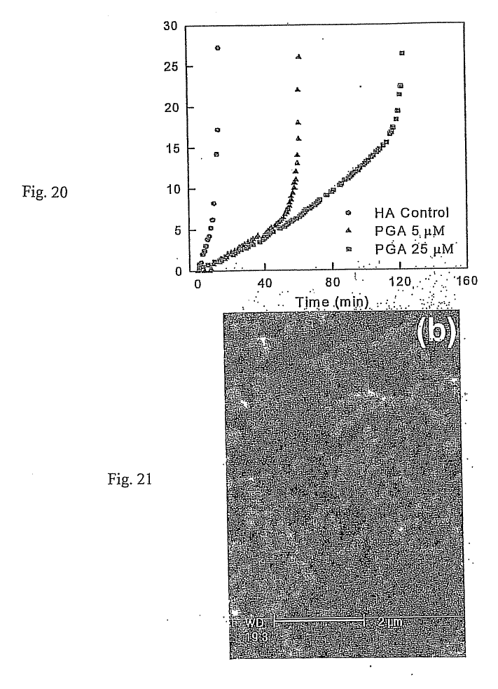

In addition to biocatalytic silicification, similar biocatalyst-mediated and localized precipitation of hydroxylapatite is desired as an approach to the formation of nanostructured restorative interphases. Following state-of-the-art HA synthesis methodology, constant composition method (CCM) was used to monitor mineralization while constant activity was maintained. In particular, the CCM method enabled evaluation of the kinetics of HA formation in the presence of a biocatalyst. A simplified polypeptide, PGA, was used as biocatalyst. Well-characterized HA seed was added to nucleate the reaction, while several process parameters were evaluated including the concentration of PGA. As a control, non-catalyzed reaction (no PGA) was also studied. The results are shown in FIG. 20.

As can be Seen from FIG. 20, Added Pga Lowered the Overall Growth Rate of Ha (seen as the time to rapid up-turn), with increased PGA concentration enhancing the effect. The ability of PGA to control the nucleation events can be seen by the prolonged duration before this sharp upturn takes place. It is worth noting that after a certain time, the ability of PGA to control HA nucleation diminishes as seen by the upturn of the curve resembling the control. Without being bound by theory, it is hypothesized that the PGA is consumed by binding to the newly formed crystal face and, once exhausted, HA precipitation proceeds at a similar rapid pace as the control (without PGA).

It should be emphasized that controlled HA precipitation is occurring only in the linear (early stage) portions of all of the experiments. PGA adsorption during HA growth is supported by the shift to longer incubation times as the concentration of the biocatalyst is increased. Without being bound by theory, it is hypothesized that the biocatalyst adsorbs onto specific crystal face(s), thus allowing the crystal growth only at other specific crystal faces (yielding needle-like or platelet-like particles) resulting in a lower overall growth rate, but with important morphology control. Wide-angle x-ray diffraction (WAXD) analysis (data not shown) showed that the synthesized HA adopts the same crystalline structure as standard HA seed, known to be dipyramidal hexagonal with unit cell dimensions: a=9.418 Å and c=6.875 Å.

### Example 6

### Silicification Within a Polymer Matrix Material

A copolymer system consisting of a 45:45:10 (wt %) composition of n-butyl acrylate, 2-hydroxyethyl acrylate (BA-HEA), and the crosslinker triethyleneglycol dimethacrylate produces a durable polymeric solid that absorbs 10 wt % water at equilibrium. A film of this copolymer was cast between glass slides separated by a 100 μm spacer and allowed to cure for several minutes under UV irradiation, following which one glass slide was removed and discarded. Isolated drops of R5 solution, as prepared in Example 1, were placed on the cured acrylate copolymer. The drops were allowed to dry slowly to enhance absorption of the R5 into the swellable acrylate, although this aspect was not confirmed. The samples were then washed with water to remove non-absorbed R5. Next, the glass-supported acrylate strip was dipped into the slightly acidic TEOS solution (as prepared in Example 1) for several minutes to allow biocatalytic silica formation near the acrylate-immobilized R5. As a control, the experiment was repeated without the incorporation of R5.

FIGS. 7a and 7b are optical micrographs of the films (a) with and (b) without immobilization of the R5 peptide in the polymer. Arrows indicate regions of silica formation. The silica islands shown in FIG. 7b are a result of shrinkage due to slow condensation polymerization and drying that occurs over the course of several hours. This is a common feature of sol-gel chemistry. The local microstructure is likely amorphous and typical of a gelation reaction.

FIGS. 7a and 7c show silica precipitation by the immobilized R5. The horizontal boundary in FIGS. 7a and 7c was defined by the droplet of R5 solution used early in the process. The R5 biocatalyst had two effects. First, the silica formation with R5 was rapid, occurring in only several minutes. Second, the precipitate was primarily localized to where the R5 was absorbed into the polymer. It appears that the R5 was both absorbed into the bulk of the BA-HEA film and adsorbed onto its surface. Surprisingly, silica formation is displayed with distinct striations perpendicular to the R5 droplet boundary, with the striations crossing the droplet boundary into a region where apparently no R5 had been absorbed.

### Example 7

### Electrospinning of Polymer-Biocatalyst Fibers

Poly(vinyl alcohol) has been used to form electrospun fibers containing poly(L-lysine) (PLL), as shown in FIG. 8. For this experiment, a 10 wt % aqueous solution of PVA was prepared by refluxing the polymer in distilled water. Dissolved PLL was added to the solution at 5 wt %, relative to PVA. This solution was pumped through a stainless steel needle (I.D. 0.26 mm) at a rate of 0.01 mL/h, using a syringe pump, with the needle held at a potential of 10 kV relative to the grounded collector positioned below the charged needle with a separation distance of 5 cm. Fibers were collected continuously on grounded aluminum foil and inspected using scanning electron microscopy (SEM). The average fiber diameter was in the 225-300 nm range. The PLL-containing PVA fibers were then exposed to silicic acid, resulting in the deposition of silica as shown in FIG. 9b.

In order to provide fibers containing silicatein, poly(ethylene-co-vinyl alcohol) (EVOH) (5-20 wt %) is dissolved in an isopropanol/water mixture at a concentration of 10% (w/v) with added silicatein at a level of 0.5 wt %. The exact polymer concentration will dictate the fiber diameter formed, with fiber jet formation occurring for Be=[η]c>1 (where [ ] is the intrinsic viscosity and c is the solution concentration). The homogeneous solutions are pumped with a syringe pump to a high voltage needle whose voltage is manipulated by a Labview™-controlled power supply so that the electrified jet's flow rate balances the volumetric flow rate of the pump. Polymeric nanofibers bearing biocatalyst are produced on electrically conductive glass slides to a total thickness of 1-10 μm.

Once prepared, the dried scaffold is stabilized by exposure to glutaraldehyde vapor (if necessary), which crosslinks the polymers through the hydroxyl groups. A portion of this material is analyzed at this reference stage with electron microscopy to determine the average fiber diameter. Finally, the scaffold is exposed to fresh silicic acid solution (described above in regard to the silica precipitation assay) by vacuum-assisted infiltration of several drops of 1 mM solution, followed by an annealing time of 10-15 minutes, following which the scaffold is washed in sodium phosphate-citrate buffer. After drying and washing the sample, various characterization methods are used to ascertain the degree and nature of silica formation. Specifically, thermogravimetric analysis (in N₂ and O₂) and FTIR of KBr pellets (co-ground with fiber) allows comparable measurements of silica content, the latter exploiting a strong absorbance between 1200-1400 cm⁻¹ for —Si—O— stretching vibrations. Environmental (low vacuum) scanning electron microscopy is used to qualitatively and quantitatively examine the silica formed, in particular the shape and amount of the silica.

### Example 8

### Localized Deposition of Polymeric Multilayers

The localized deposition of polymeric multilayers allows the formation of two-dimensional (2D) scaffolds. The microfluidic channels (μFNs) are filled by a liquid carrying the reactants under a flow. The targeted substrate being sealed with the μFN, and the reactants flowing through the microchannels, the substrate's surface is further derivatized. Here, microfluidic channels are used to achieve such micropatterns with high line resolution, in combination with the LbL deposition under laminar flow. Elastomeric molds serve as the microfluidic network after processing with the soft lithographic methods developed for microcontact printing of self-assembled monolayers (SAMs). PDMS precursor is cast against a photolithographically prepared master that contains a pattern complementary to that to be reproduced (FIG. 10).

In the present case, the patterned photoresist consists of an array of 20 parallel lines, 12 μm thick, 34 μm wide, separated by 34 μm, 2.36 mm long and connected to a 5×10 mm² pad at each end: the pads defined outlines for reservoirs to which fluid could flow. The flow of a solution in small channels is afforded by a mechanical syringe pump. The multilayer patterning is performed in two steps: polymer solution is first allowed to fill the microfluidic channels, which are formed by contact between an elastomeric mold and a hydrophilic substrate, and subsequent removal of the weakly bonded polymer on the surface by channel flushing. The flow self-assembly process (FSA) for a single bilayer (to be repeated multiple times) consists first of flowing a polystyrene sulfonate sodium salt (PSS) solution for anionic deposition onto a charged surface through the microfluidic network, then flushing, and finally flowing of poly(allylamine hydrochloride) (PAH) for polycation deposition.

The SSA processing route is capable of growing polymer multilayers with great thickness resolution, as revealed by UV absorbance and atomic force microscopy (AFM), that is proportional to the layer thickness. This z-axis resolution is exploited by adsorbing during the SSA processing selected biocatalyts within 100 nm-thick nanostratified scaffolds at one of three vertical positions: bottom, middle, and top.

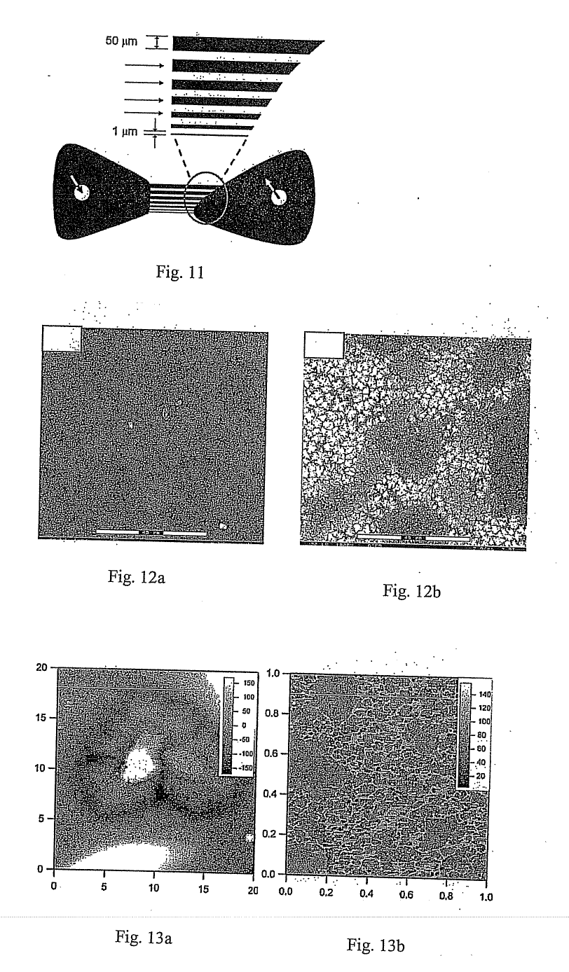

For the mineralization studies on nanostratified microlines, multilayered lines of PSS and PAH as discussed above in reference to FIG. 10 are first processed. A particular microfluidic network, shown in FIG. 11, has been designed, in which with one experiment a range of scaffold widths is created, ranging from 50 to 1 μm. In order to maintain a similar pressure drop for each channel, and thus similar flow velocity, the length is in proportion to the channel dimension squared. As in the case of the SSA-prepared 1D scaffolds, enzymes from the Wood laboratory are incorporated at various depths within the polyelectrolyte multilayers. Once dried, the scaffolds are exposed to silicic acid to promote the deposition of silica.

### Example 9

### Layer-by-Layer (LbL) Adsorption

The LbL process is illustrated by the following example, in which three multilayer films were formed. In one multilayer film, polystyrene sulfonate sodium salt (PSS), molecular weight (MW)=70 kDa, was used as the host polyanion with poly(L-lysine) (PLL) as the polycation. Twenty PSS-PLL bilayers, each about 1.5 nm thick, were spin coated onto a quartz disc to provide a first, twenty-layer multilayer film that will be referred to as (PSS-PLL)₂₀. A second multilayer film was formed from nineteen bilayers of PSS and poly(allylamine hydrochloride) (PAH), with one bilayer of PSS/PLL as an outer layer. A third multilayer film using nineteen bilayers of PSS-PAH (no PLL) was used as a control. This design produced three films each about 35 nm thick, with biocatalyst (PLL) uniformly distributed throughout the host (the first multilayer film), on the surface of the host (the second multilayer film), or absent (the third multilayer film).

A silicic acid precursor solution was prepared from tetramethyl orthosilicate, Si(OCH₃)₄. The solution was applied in drops onto the three LbL films, maintained in a moist environment, then dried at room temperature under vacuum for 24 hours. Samples were characterized using SEM, AFM, FTIR and nano-indentation as described below.

Representative scanning electron micrographs (SEM) from the non-catalyst control (FIG. 12a) and (PSS-PLL)₂₀ (FIG. 12b) show that there was essentially no reaction product on the non-PLL control (FIG. 12a). One layer of PLL produced a relatively thin layer of silica (not shown). The film containing PLL throughout its depth, (PSS-PLL)₂₀ (FIG. 12b), exhibited distinct silica formation. The “snowflake” or dendritic appearance is clear at higher magnifications (not shown), suggesting a crystalline form of silica. The extent and morphology of the formed silica varied within the silicified regions of the (PSS-PLL)₂₀ sample, with continuous layers forming in some areas.

The topography of silicified and non-silicified areas were evaluated with atomic force microscopy (AFM, Asylum Research MFP-3d). The non-silicified areas were extremely flat (±2 nm), indicative of LbL spin coating onto flat quartz discs. The dendritic or snowflake regions were readily imaged and found to be approximately 300 nm thick as shown in FIG. 13a. The AFM phase image (FIG. 13b) shows nanoscale particles within a larger continuous phase.

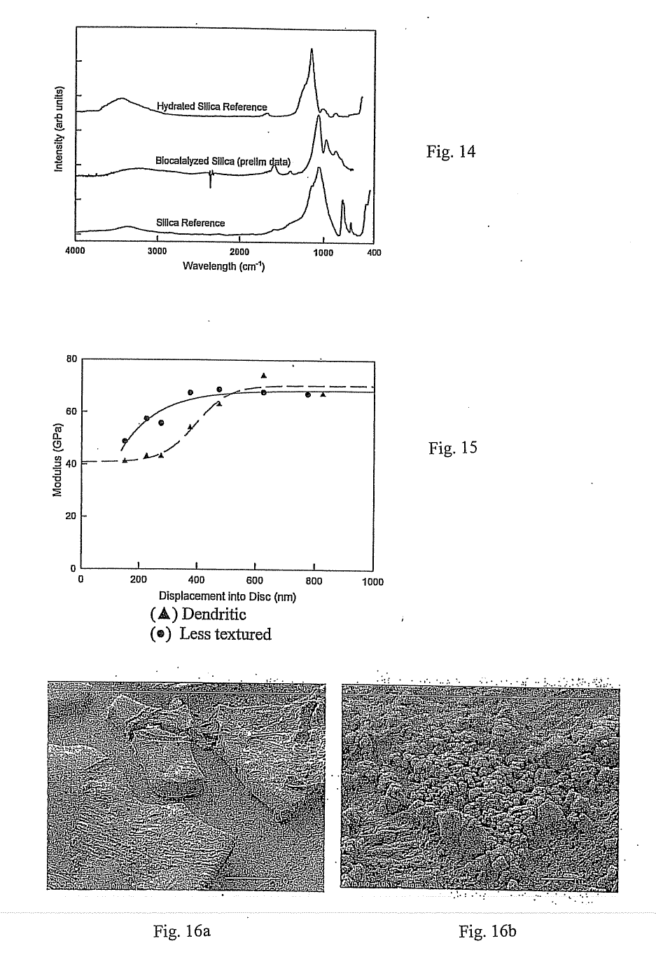

FTIR can potentially confirm the presence of different hydrated forms of SiO₂. However, as the multilayer film samples were formed on either quartz discs or silicon wafers, standard transmission modes were not possible. Two approaches to developing methods free of artifacts from the substrates were attempted. First, a polyimide tape (KAPTON™, DuPont) was placed over the quartz disc, followed by the LbL and silicification procedures. The tape carrying the silicified sample was removed from the disc and examined with transmission FTIR. In a second approach, the formed silica on the quartz substrate was examined directly in reflectance mode using an ATR (attenuated total reflectance) objective (Bruker, Tensor 27 FTIR). With both procedures control FTIR spectra were obtained from (PSS-PLL)₂₀ non-silicified surfaces and the untreated quartz discs. Reflectance FTIR was able to differentiate the formed silica from both the untreated fused quartz disc and the (PSS-PLL)₂₀ applied to the quartz disc but not silicified. Most importantly, the silicified surface reasonably matched the standard spectra for silica (Y #169, Bio-Rad Laboratories) as shown in FIG. 14. The approach with polyimide tape also was useful. Films can also be formed directly onto commercial-grade aluminum first surface mirrors, which should enhance the FTIR reflectance signal.

The hardness and modulus of (PSS-PLL)₂₀ silicified samples were determined with an MTS Nano-Indentor XP. Nano-indentation allows determination of mechanical properties as a function of depth at and near the surface. Two different loading programs were selected from the available system software (TestWorks™, MTS). “XP Basic Hardness, Modulus, Load Control” allowed indentations at specified depths, while “CSM Hardness, Modulus” provided continuous depth profiling. Preliminary studies were conducted to determine the appropriate inputs for each program. A Berkovich indentor and the XP head were used in all tests. Tests were conducted on the as-received 1″× 1/16″ quartz discs (Chemglass, CGQ-0600-01) that are used as substrates; discs layered with (PSS-PLL)₂₀; and (PSS-PLL)₂₀ discs silicified in several circular areas, each approximately 5 mm in diameter. Within the silicified areas both dendritic and regions with less distinct morphological features could be viewed and tested with the nano-indentor. Calibration was confirmed before each series by indenting the MTS fused silica standard.

The modulus and hardness of the untreated Chemglass quartz disc were 73.8±2.2 and 10.8±1.1 GPa, respectively, consistent with the literature. The indentation profiles of the (PSS-PLL)₂₀ non-silicified samples were comparable to the untreated disc, although the CSM procedure detected a small increase in the hardness vs. depth profile at about 35 nm displacement into the surface, consistent with the 35 nm thickness of the polymer/catalyst multilayer (data not shown). Nano-indentation of the formed silica provided quite interesting results. FIG. 15 shows plots of the modulus versus displacement into the surface of the dendritic (dashed line) and less-textured regions (solid line) of the silicified (PSS-PLL)₂₀. The values for the dendritic areas were about 40 GPa for the first 300 nm, then increased to a plateau value (approximately 70 GPa) equal to the measured modulus of the untreated substrate quartz disc. AFM showed the dendrites to be about 300 nm thick (FIG. 13, left). The consistency with the AFM data indicates that the formed silica is being accurately measured by nano-indentation and that its modulus is about 40 GPa. Hardness (not shown) increased monotonically near the surface from about 0.6 to 1.0 GPa.

Comparable nano-indentation tests were conducted on a representative particulate-reinforced dental composite. Sculpture Plus™ (Pentron Corp.) is a dimethacrylate-based composite with 75 wt % glass particles having an average size of 1 μm. Cylindrical samples were prepared with a polyvinyl siloxane mold and glass cover slide. The sample was cured (Cure-Lite™ Plus, Pentron Corp.) through the glass slide for 9 minutes, removed and cured an additional 15 minutes. The sample was embedded in epoxy and metallurgically polished through 0.05 μm alumina abrasive. The modulus measured with nano-indentation was 14-16 GPa, consistent with the manufacturer's reported value of 13.3 GPa. The measured hardness was 0.8-1.0 GPa (about 80-100 kg/mm²), which is consistent with the hardness values for contemporary composites reported in the literature of 50-70 kg/mm².

In summary, the modulus and hardness of the silica formed using PLL were about 40 GPa and 0.8 GPa, respectively, while the values for a typical commercial composite were 15 GPa and 0.9 GPa. Use of the more complex R5, and particularly the engineered full protein catalysts is expected to provide more control over morphology and even higher mechanical properties. The fact that the formed silica had about three times the modulus of the composite indicates that it can be possible to form a wear-resistant surface on dental restorative materials.

### Example 10

### Localized Mineralization on Surfaces—PEI Catalyzed Silicification

Translating biocatalyzed mineralization to a useful dental restorative interphase requires the ability to form controlled films or coatings on surfaces. One likely clinical scenario would be the adsorption of a biocatalyst onto a surface with subsequent application of a mineral precursor. As a means to deposit the biocatalysts in a well-controlled manner, a rapid layer-by-layer (LbL) method, or polyelectrolyte spin assembly (PSA) wherein polymeric monolayers of alternating charge (one a “carrier”, the other a biocatalyst) were sequentially adsorbed onto a substrate using a conventional spin-coater. This method allowed concise control of film thickness and position of the catalyst relative to the surface.

Polystyrene sulfonate (PSS), which is analogous to a resin used in dental desensitizing agents, was used as the “carrier” polyanion (and thus corresponds to the polymer matrix) and PEI biocatalyst as the polycation. A single layer of PEI was adsorbed onto the disc by spin coating. Several other multilayer arrangements and controls were also evaluated. Silicic acid, or Si(OH)₄, was prepared from TMOS precursor solution and applied to the PSS/PEI film. Samples were characterized with SEM, AFM, transmission electron microscopy (TEM), and attenuated total reflectance Fourier transform infrared (ATR-FTIR).

It was found that PEI was quite effective in catalyzing silica formation from either the parent TMOS or silicic acid. FIG. 21 shows the dense network that formed at the PEI surface when exposed to silicic acid, noting that a similar result also occurred from TMOS. It was confirmed that the material formed is, compositionally, silica, and the disordered structural appearance suggests amorphous silica. It is worth noting that as a comparison, silica formed on (PSS/PLL)₂₀ multilayers obtained in Example 9 was in crystalline form.

### Example 11

### Localized Mineralization on Surfaces—Biocatalyzed Hydroxylapatite (HA)

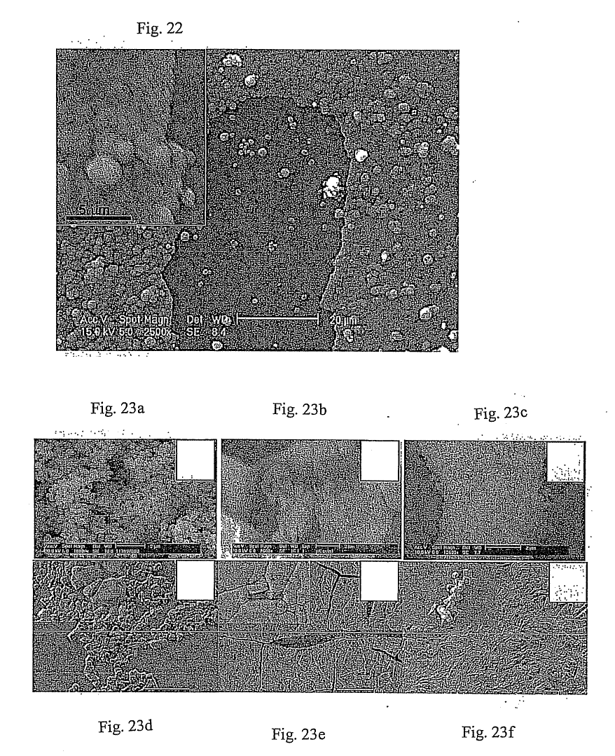

In this study, the PSA surface preparation method was used to coat a silicon wafer with PGA within a PEI-(PGA-PAH)₅-PGA construct (PEI first layer, followed by 5 layers of alternation of anionic PGA and cationic carrier polyallylamine hydrochloride (PAH), and terminated with PGA). These substrates were then immersed in simulated body fluid (SBF), which is used as the mineral precursor, at 37° C. for a period extending to 1 week. FIG. 22 shows dramatic and complete surface coverage with a mineral, which detailed composition is yet to be determined. Nevertheless, we postulate that it is an amorphous calcium phosphate. Etching the surface with a razor (FIG. 22, center) revealed finite and uniform depth of the HA coating, while higher magnification inspection revealed that the coating was an agglomeration of primarily spherical particles with diameters in the range of 200 to 900 nm. Considering the small characteristic dimension of dentin tubules, the nanoscale nature of PGA-nucleated HA can provide great benefits for use as dental materials.

### Example 12

### Dimethacrylate-Based Dental Resin as the Polymer Host

Because biocatalysis within polymer hosts under ambient conditions is itself a novel technology, up to this point we designed systems that optimized the likelihood of successfully forming silica. For example, polyvinyl alcohol, and poly(ethylene-co-vinyl alcohol) were used because of their ability to form high surface area electro-spun fiber meshes (Example 7). Butyl acrylate-2-hydroxyethyl acrylate (BA-HEA) was used because the crosslinked polymeric solid absorbs water, thus enhancing absorption of the catalyst solution (Example 6). Polystyrene sulfonate allowed use of the precise layer-by-layer process to control positioning of the poly(L-lysine) catalyst within the host (Example 9). This strategy was a necessary first phase of the work.

To study the feasibility with practical dental clinical systems, a commercial dimethacrylate-based resin (Bond-1™, Pentron) was used to host the silicification reaction. Bond-1™ contains glycerol dimethacrylate, 2-hydroxy ethyl methacrylate (HEMA) and other dimethacrylates in an acetone/ethanol carrier. Bond-1™ is a dentin adhesive and not a composite, but was selected because its entire surface would be available for the reaction, enhancing subsequent detection of silica.

Six or seven drops of as-received Bond-1™ were spin-coated for 30 seconds at 5000 rpm on a cleaned 1 cm×1 cm silicon wafer and then visible light-cured for one minute. A second layer was similarly applied. Four layers of poly(L-lysine) were each applied to the cured Bond-1™ host and spin-coated for 10 seconds at 5000 rpm. The silicification reaction with pre-hydrolyzed TMOS precursor solution was conducted as described above. The sample was coated for, and examined with SEM.

As with the solution reaction, the BA-HEA thin film, and the layer-by-layer process (previous examples) different morphologies were observed. FIG. 16a is an SEM of 1 μm thick layers that were found throughout much of the silicified regions. At higher magnification (not shown) the layers appear to have a folded texture on the surface. Angular particles approximately 0.2-1.0 μm in size (FIG. 16b) were often found in clusters and may have nucleated on the textured layer. These angular particles would be particularly useful for sealing dentin (tooth) tubules, improving the interface between a restorative material and the tooth and/or more effectively sealing the tooth, thereby decreasing sensitivity and potentially improve long-term stability of the restorative material.

### Example 13

### Silicification of Composite Materials

Silicification experiments were conducted on visible-light cured (VLC) commercial dental composites (SCULPTURE™, Pentron Corp.). The cured composite samples received no pre-treatment, or washing with 100 mM NaOH or with 100 mM HCl to hydroxylate the surface. Solutions of PLL were applied to the surface and dried, followed by silicification. Well-defined, continuous layers were formed that appeared to subsequently fragment. Control samples without PLL resulted in an ill-defined morphology. Modulus and hardness of the composite from nano-indentation tests were the as-expected 14.6-19.7 GPa and 0.87-1.1 GPa, respectively. Values for the silicified surface were 10.6-14.5 GPa and 0.15-0.89 GPa, respectively.

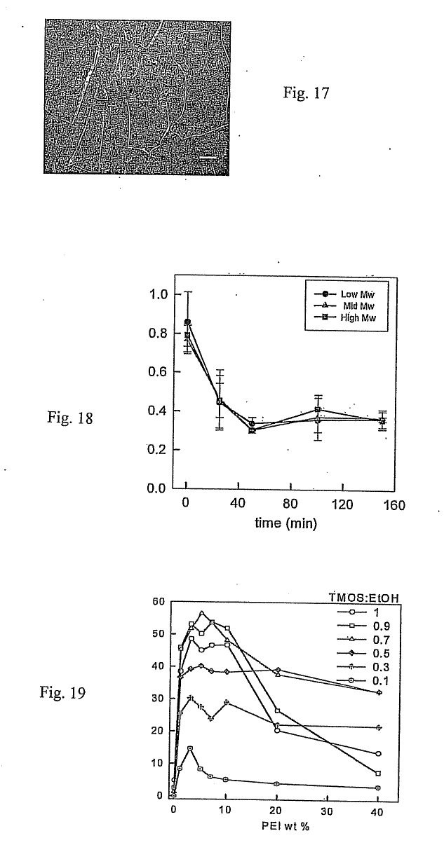

To determine the effect of VLC on catalyst reactivity, solutions of PLL were adsorbed onto uncured composite by coating the composite onto the surface of the composite. The composite was then cured using a visible light source, and silicified with 100 mM hydrolyzed TMOS at pH of 5 or 7. As above, continuous layers of silica were formed that subsequently fragmented (FIG. 17). Visible light curing apparently did not alter the biocatalyst (PLL) nor the availability of reactive species that promote the aggregation and direct the formation of silica. This result demonstrates the use of uncured dental acrylates as carriers of mineral catalysts as part of the various clinical applications.

### Example 14

### Miscibility and Interactions of Silicification and Polymerization Chemistries

In the above examples, the biocatalyst and precursor were applied on model surfaces and dentin in separate steps. For practical dental applications it would be useful to incorporate the mineralization and resin polymerization chemistries. In this example, mixing biocatalyzed mineralization and visible-light curing (VLC) resins was studied.

Prehydrolyzed 100 mM TMOS in 0.5 ml ethanol and 3.0 ml Tris-HCl was reacted with 10⁻³M low molecular weight PLL at 1:2 v/v % to make solution 1. To solution 1, 0.008 g of DL-camphorquinone (CQ), 0.032 g of p-dimethylaminobenzoate (EMBO), 0.3 g of 2-hydroxyethyl methacrylate (HEMA), and 1.0 ml of anhydrous ethanol were added to make solution 2. Separately 0.002 g of CQ, 0.008 g of EMBO and 0.01 g of γ-methacryloxypropyl-trimethoxysilane (MPTS) were added to solution 1 to make solution 3. Several minutes after addition, the combined solutions turned cloudy and produced a white precipitate. Without the PLL, the precursor solution combined with HEMA or MPTS was not as cloudy. This finding demonstrates that the PLL biocatalyst can promote the condensation and flocculation of the silicic acid precursor in the presence of HEMA and other constituents commonly found in visible-light curing dental resins.

Solutions 2 and 3 containing the precursor, catalyst, and VLC constituents were brushed onto both sides of two dentin disks and cured by visible light for about 5 minutes. The mixtures appeared to harden visually as routinely observed with VLC resins. The hydraulic conductance was then measured. In both disks, the permeability decreased significantly (>50%).

### Example 15

### Mineralization on Biocatalytic Multilayer Films

Silicon wafers (Wafernet Inc., San Jose, Calif.) were etched with “piranha acid” (30:70 v/v of 30% H₂O₂ and concentrated H₂SO₄) to produce hydroxylated surfaces prior to deposition. To produce the first layer with a positively charged surface, low molecular weight PEI (Sigma-Aldrich, 4 wt % solution) was deposited by dipping the substrate for 15 minutes. Subsequently, multilayers were formed by alternating deposition of low molecular weight PLL (Sigma-Aldrich, MW=30,000 to 70,000 Da, 0.01 M based on repeat unit) followed by PSS (Sigma-Aldrich, MW=50,000 to 60,000 Da, 0.01M) for 15 minutes each, rinsing with de-ionized water after each layer deposition. PEI also was used as the polycation biocatalyst in the multilayer films. The LbL process was repeated until multilayer films of PEI-(PLL/PSS)₁₀, PEI-(PEI/PSS)₁₀ and PEI-(PEI/PSS/PLL/PSS)₁₀ were formed.

An alkoxide precursor solution was prepared as follows. Tetramethyl orthosilicate (TMOS) was prehydrolyzed with 0.01 M HCl and citrate-phosphate buffer to the final concentrations of 0.01 M, 0.1 M and 0.2 M, respectively. Each of these precursor solutions was then added dropwise onto the catalytic multilayer films and the samples were incubated in a water chamber to control relative humidity and promote hydrolysis. After 24 hours, the samples were dried in a vacuum at room temperature.

For the PEI-(PLL/PSS)₁₀ films the effects of precursor concentration (10 mM to 200 mM), solvent (buffer to ethanol ratio) and drying temperature (60° and 150° C. for 1 hour) on the formed silica coating also were investigated. As a control, TMOS solution was applied to an acid-etched wafer with no catalytic multilayer film. Morphology was investigated by optical microscopy (Leica, Bannockburn, Ill.), SEM (JEOL USA Inc., Peabody, Mass.) and AFM (Asylum Research MFP-3d, Santa Barbara, Calif.). The average surface roughness (rms) and contact mechanical stiffness of the silicified PEI-(PLL/PSS)₁₀ films were also determined using AFM. For each sample, a 5 μm topographical AFM image was taken and a subsequent series of 16×16 force curves were performed area-wise.

The morphology of the formed silica coatings are compared across the three LbL catalytic multilayer films (FIGS. 23d-f) and to the morphology of silica catalyzed in solution (FIGS. 23a-c) with the same polyelectrolytes. The silica formed on the PEI-(PLL/PSS)₁₀ film was plate-like (FIG. 23e), while the PEI-(PEI/PSS/PLL/PSS)10 film produced a silica consisting of dendritic networks, plate-like and spherical particles with average particle sizes of 1 μm to 10 μm and >1 μm, respectively (FIG. 23f). Silica produced on the PEI-(PEI/PSS)₁₀ multilayer films consisted of dendritic networks with average particle size of about <1 μm to 5 μm. Without a catalyst, the formed silica was fine and gel-like (not shown). Thus, the catalyst on the surface provided control over the morphology of the silica. In the presence of a catalyst, the silica coatings were coherent (FIGS. 23e-f) except for PEI-(PEI/PSS)10, which mostly formed a dendritic network of particles (FIG. 23d).

In solution, PEI formed nano-scale particles that agglomerated into clusters (FIG. 23a). PLL-catalyzed silica was predominantly plate-like in solution (FIG. 23b), similar to the morphology that formed a coherent coating when catalyzed onto the surface (FIG. 23e). The combined PEI/PLL catalyzed silica produced a non-homogeneous mixture of plate-like and spherical particles in solution (FIG. 23c), somewhat in contrast to the dendritic individual structures of the surface-catalyzed silica that formed a coherent coating (FIG. 23f).

In another study, the silica yield with PLL was lower than that obtained with either PLL and PEI together or PEI alone. As discussed above in Example 4, PEI aqueous solutions are capable of both hydrolysis and condensation of TMOS (data not shown). This indicates that the hydrolysis and condensation of the precursor is specific to the catalyst. Thus, presence of additional macromolecules in the vicinity of PLL during catalysis of silica, as found in marine proteins such as silaffins, seems to influence the role that long chain polyamines play in promoting silicification.

### Example 16

### Surface Topography of Minerals on Biocatalytic Multilayer Films

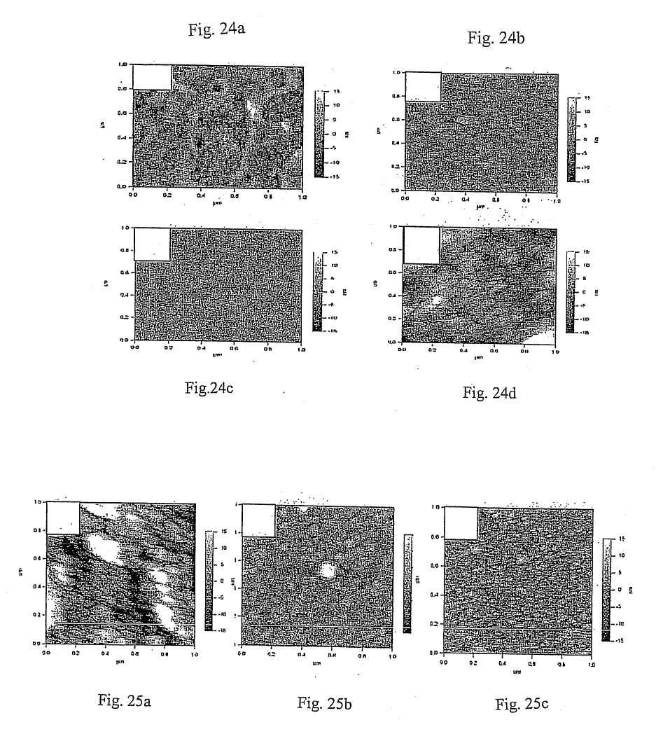

Atomic Force Microscopy (AFM) was used to characterize the surface topography of silica catalyzed by PEI-(PLL/PSS)₁₀ films and non-catalyzed silica coatings as described in Example 18. FIG. 24 shows four high resolution images, each 1 μm on a side with a vertical scale of contrast from black to white representing 115 nm in height. Topographically, the silica formed on PEI-(PLL/PSS)₁₀ multilayer films consisted of spherical particles, 0.01 μm to 1 μm in size. A mesoporous microstructure was evident for silica catalyzed from low, 10 mM, TMOS precursor concentrations in buffer (FIG. 24a) while a more homogeneous silica structure formed from 200 mM TMOS in buffer (FIG. 24b). Silica catalyzed from TMOS in ethanol, on the other hand, exhibited features of similar lateral dimensions but much smoother, sub-nm roughness (FIG. 24c) compared to non-catalyzed silica in the same solvent (FIG. 24d). This shows that the concentration of silicon precursor or the solvent used in the reaction allows further control of the surface morphology, similar to effects observed for the formation of silica on silicatein filaments.

AFM imaging also was used to examine the effect of drying (FIG. 25). The catalyzed silica consisted of smaller particles and thus less roughness (FIG. 25(b)), compared to non-catalyzed silica (FIG. 25a) when dried at room temperature. The particle size at the surface seemed to decrease with drying at 150° C. (FIG. 25c). For traditional sol-gel produced thin films, drying and heating promote syneresis or the collapse of pores leading to structural relaxation and densification. The present data demonstrate similar results with biocatalyzed silica film indicating that drying could be used to affect silica morphology.

The results presented indicate several possible mechanisms of silica catalysis by biocatalysts within polymer hosts. First, the mere presence of the catalyst influences the rate of silicification and the properties of the formed silica. Second, silicification mediated by these biocatalysts can be catalyst specific. As films, the deposited silica is stabilized by the chemistry and orientation of the biocatalysts on the surface thus leading to optimized geometries on or within the multilayered polymer host. Third, these biocatalysts can complement each other or act synergistically as in the case of larger particles formed with combined PEI and PLL catalysis.

Contact mechanical stiffness measurements revealed the mechanical properties of these films to be minimal. The average stiffness when contacting silica particles was 40 N/m, as compared to about 48 N/m for the non-silicified polyelectrolyte film. The low values suggest that the silica mineral formed on the multilayer organic film can be hydrated and cannot be completely crystalline.

Table 1 shows the RMS roughness (nm) of selected samples determined with AFM at different scan sizes. The catalyzed samples were consistently smoother than the non-catalyzed samples for all combinations of processing conditions. For the catalyzed samples, increasing the drying temperature to 60° C. decreased roughness with either the buffer or ethanol solvent. Both of these observations were consistent with the SEM examination (FIGS. 24 and 25). Ethanol alone may have resulted in lower roughness, although this could not be separated from the drying effect. Clearly, the presence of the catalyst, drying temperature, and type of solvent affect the topography and roughness of the surface.

TABLE 1

| | | Drying | Scan Size, μm | | | |
|---|---|---|---|---|---|---|
| | Solvent | Temp. | 15 | 7 | 5 | 1 |
| Non-catalyzed | Buffer | RT | 12.19 | 12.72 | 12.78 | 8.89 |
| Non-catalyzed | Buffer | 60° C. | 27.63 | 16.54 | 14.79 | 6.37 |
| Non-catalyzed | Ethanol | 60° C. | 11.42 | 11.19 | 6.48 | 3.75 |
| Catalyzed | Buffer | RT | 7.06 | 6.04 | 5.63 | 3.04 |
| Catalyzed | Buffer | 60° C. | 1.86 | 2.061 | 2.34 | 2.39 |
| Catalyzed | Ethanol | 60° C. | 1.21 | 1.08 | 1.07 | 0.75h |

When applied to a surface within a multilayered film, PLL and PEI maintained their ability to catalyze silica from pre-hydrolyzed TMOS solutions. The type of biocatalyst, the precursor concentration, the precursor solvent, and drying temperature all affected the morphology of the formed silica.

The terms “a” and “an” herein do not denote a limitation of quantity, but rather denote the presence of at least one of the referenced item. All ranges directed to the same quantity or property are inclusive of the endpoint and independently combinable. The modifier “about” used in connection with a quantity is inclusive of the stated value and has the meaning dictated by the context (for example, includes the degree of error associated with measurement of the particular quantity). All cited patents, patent applications, and other references are incorporated herein by reference in their entirety.

While the invention has been described with reference to a preferred embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A dental material composition, comprising
a polymer matrix material; and
a biocatalyst that promotes the deposition of a mineral from a mineral precursor.

2. The composition of claim 1, wherein the biocatalyst is a polyamine, a polypeptide containing at least 10 amino acid or amino acid analog residues, an enzyme, or an enzyme fragment effective to promote the deposition of a mineral from a mineral precursor.

3. The composition of claim 2, wherein the biocatalyst is poly(L-lysine), poly(ethyleneimine), poly(glutamic acid), or a combination comprising at least one of the foregoing polyamines.

4. The composition of claim 2, wherein the biocatalyst is R5, an analog of R5, silaffin, an analog of silaffin, or a protein derived by DNA shuffling of silaffin.

5. The composition of claim 3, wherein the biocatalyst is a silicatein.

6. The composition of claim 1, wherein the biocatalyst is in the form of a layer in a polycation/polyanion multilayer film.

7. The composition of claim 6, wherein the multilayer film is poly(ethyleneimine)-(poly(L-lysine)/poly(styrene sulfonate))ₘ, poly(ethyleneimine)-(poly(ethyleneimine)/poly(styrene sulfonate))ₙ, or poly(ethyleneimine)-(poly(ethyleneimine)/poly(styrene sulfonate)/poly(L-lysine)/poly(styrene sulfonate))ₚ, wherein m, n, and p are independently an integer of 2 to about 20.

8. The composition of claim 1, in the form of dental adhesive, a dental sealant, a dental restorative material, a dental desensitizer, an orthodontic wire, an orthodontic bracket, or a dental implant.

9. A method of forming a dental material, comprising
combining a biocatalyst that promotes the deposition of a mineral and a polymer matrix material to form a biocatalyst-polymer composite.

10. The method of claim 9, wherein the biocatalyst-polymer composite is contacted with a mineral precursor in an amount and for a time effective to deposit a mineral.

11. The method of claim 10, wherein the biocatalyst-polymer composite is contacted with a ceramic precursor in an amount and for a time effective to deposit a ceramic, forming a biocatalyst-polymer-ceramic composite.

12. The method of claim 11, wherein the ceramic is silica or a calcium phosphate.

13. The method of claim 12, wherein the calcium phosphate is hydroxylapatite.

14. The method of claim 9, wherein the biocatalyst is within the polymer matrix material.

15. The method of claim 9, wherein the biocatalyst is within and/or on a surface of the polymer matrix material.

16. The method of claim 9, wherein the polymer matrix is a template for the deposition of the mineral.

17. The method of claim 9, wherein the polymer matrix is a polymeric nanofiber.

18. The method of claim 9, wherein the polymeric matrix is a layer of an anionic polymer.

19. The method of claim 9, wherein the biocatalyst-polymer composite is a polycation/polyanion multilayer film.

20. A dental treatment method, comprising
applying the biocatalyst-polymer composite of claim 1 to a dental site to be treated; and
contacting the biocatalyst-polymer composite with a mineral precursor in an amount and for a time effective to deposit a mineral, forming a biocatalyst-polymer-mineral composite.

21. The method of claim 20, wherein the mineral is deposited as a layer.

22. The method of claim 20, wherein the mineral is deposited in a gradient.

23. A dental restoration formed by the method of claim 21.

24. A method of forming a dental restoration, comprising
applying a biocatalyst-polymer-mineral composite to a dental site to be restored.

25. The method of claim 24, wherein the mineral is silica or a calcium phosphate.

26. A dental restoration formed by the method of claim 25.


SEQUENCE LISTING (11 sequences)

SEQ ID NO: 1 - DNA, 1360 bp, Tethya aurantia
atcgaatcaaatcgtgagtattgctccgagttccaaggaaagtgaaagcaaaggattccagctacagtaaagatgtatctcggcacgttggttgttttgtgtgttttgggggctgctattggagagccaatgcctcagtatgagttcaaggaggaatggcagctgtggaagaaacaacatgacaagtcttacagcaccaacttggaggaactggagaaacatcttgtctggctctccaacaagaagtacattgaactgcacaatgccaatgcagacacctttggattcactctagctatgaaccatctaggagatatgactgaccatgaatacaaggagagatacctcacatacactaacagcaaatctggtaactacaccaaggtgttcaaacgtgagccatggatggcctacccggagactgtagattggagaacaaagggcgctgtgactggtatcaagagccagggagattgtggtgccagctatgcattcagtgccatgggtgcacttgaaggaatcaatgcacttgctactggaaagctgacctatctcagtgaacagaacatcattgattgctctgtaccttatggtaaccatggttgcaagggtggaaacatgtatgtggctttcctctatgttgttgctaacgaaggagttgatgatgggggttcctatccatttagaggaaagcaatccagttgtacgtatcaagagcagtaccgtggtgcaagtatgtctggctcagttcaaatcaacagtggtagtgaatctgatctggaagcagctgtagccaatgttggtccagttgcagtagctattgatggagagtcaaatgctttcagattctattacagtggagtgtacgactcctccagatgttctagtagcagtctcaaccacgccatggtgatcactggctatggaatttcaaataaccaggaatactggcttgcaaagaacagctggggtgagaactggggagaactgggctatgtgaagatggccaggaacaagtacaatcaatgtgggattgctagtgatgcctcctaccccactctctagcatgtcagccagcccagtctgaaactgaactagaattatcaatagttaaataactgtgtgttttatacatgtgaacaatagacttgatcatcctttagtaagtattatatgtgatgagtgtttgtccaatccaacattagctctgacatgtaaattattgtaataatgattctgtgattatgtctcaatgattattgtattcacaatggcatctaatttgtatacaagcccctcaatcactgactgatctcattataatttatttgatggactacaaaaaaaaaaaaaaaaaaaa

SEQ ID NO: 2 - DNA, 1418 bp, Tethya aurantia
gagttagctaattctattgtaagaactagacttcagttagtaatcactaccaagtgtgttgttgagagtggctaaggagagatgaagtggattgtagctgtttgtatggttggatttctggtggctgtgacaagtgcaggccgtcaccagccagtgtttgaacaccatgaagaatggcagctgtggaagagtcaacatggcaagtcgtaccgttcagggctgcaggagctggagagacacttggtgtgggtctctaacaaagagtacattgacagacacaatgcaaatgcagatgtgtttggattctcactagccatgaaccactttggagacctgtctgataatgagtttgtggacaagtacttgagctacaccaagtctgacaagaagaagagaaatgtgaagatgtttgaggctccagagggagtcagctacccagagtcgctggactggagaacaaagggagctgtgacctctgtcaagaaccaaggagactgtggtgctagttatgcattcagtgctatcggatctttagaaggagcactctctttggcacaaggaaaactcacctacctcagtgaacagaatgtcattgactgctcagttgcatatggtaaccatggatgccagggaggtaacatgtacaacacatatctgtacattctctccaacgatggaattgacacatccgatggttatccattcaaaggcaagcaaacttcctgcacgtatgacagatcatgtcgtggtacttcaatatctgggagcattgctatcacgagtggaagtgagtctgacctccaggcagctgtagccagtgctggaccagtggctgttgcagtggatgggtcaagcagagctttcaggttttatgattatggtctttataatcttcctggatgctcaagctaccagctgtcccatgctcttctgattacaggctatggtagcttcaatgggaatcaatactggcttgtcaagaatagctggggtactaactgggggatgagtggctacatcatgatgacaagaaacaactacaaccaatgtggtattgcaactgatgccgcctacccaaccttgtagcagtcagatgaaactctttagcttgaaaaaaactgttataacttgcaatcagtttatgtaactctccatttcatcactcgtactgtggtgttatcgtaatgtatgtgttagactaggctagacgttagacttgcattcctctgattggtagcacctgtttttttctccagtccttgaaatcctttgtgtctttgaaaatgtataaatctttgaaagtgttgcgtcacaaactctcttacatagcaataccatgtgtgtctctttgtgctcttcaattgttgcactttgcacaccatgtcattcaattcaccttccaaaaaaaaaaaaaaaaa

SEQ ID NO: 3 - protein, 330 aa, Tethya aurantia
MYLGTLVVLCVLGAAIGEPMPQYEFKEEWQLWKKQHDKSYSTNLEELEKHLVWLSNKKYIELHNANADTFGFTLAMNHLGDMTDHEYKERYLTYTNSKSGNYTKVFKREPWMAYPETVDWRTKGAVTGIKSQGDCGASYAFSAMGALEGINALATGKLTYLSEQNIIDCSVPYGNHGCKGGNMYVAFLYVVANEGVDDGGSYPFRGKQSSCTYQEQYRGASMSGSVQINSGSESDLEAAVANVGPVAVAIDGESNAFRFYYSGVYDSSRCSSSSLNHAMVITGYGISNNQEYWLAKNSWGENWGELGYVKMARNKYNQCGIASDASYPTL

SEQ ID NO: 4 - protein, 334 aa, Tethya aurantia
MKWIVAVCMVGFLVAVTSAGRHQPVFEHHEEWQLWKSQHGKSYRSGLQELERHLVWVSNKEYIDRHNANADVFGFSLAMNHFGDLSDNEFVDKYLSYTKSDKKKRNVKMFEAPEGVSYPESLDWRTKGAVTSVKNQGDCGASYAFSAIGSLEGALSLAQGKLTYLSEQNVIDCSVAYGNHGCQGGNMYNTYLYILSNDGIDTSDGYPFKGKQTSCTYDRSCRGTSISGSIAITSGSESDLQAAVASAGPVAVAVDGSSRAFRFYDYGLYNLPGCSSYQLSHALLITGYGSFNGNQYWLVKNSWGTNWGMSGYIMMTRNNYNQCGIATDAAYPTL

SEQ ID NO: 5 - DNA, 798 bp, Cylindrotheca fusiformis
atgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaa

SEQ ID NO: 6 - protein, 15 aa, Cylindrotheca fusiformis
SSKKSGSYSGSKGSK

SEQ ID NO: 7 - protein, 18 aa, Cylindrotheca fusiformis
SSKKSGSYSGYSTKKSGS

SEQ ID NO: 8 - protein, 19 aa, Cylindrotheca fusiformis
SSKKSGSYSGSKGSKRRIL

SEQ ID NO: 9 - protein, 485 aa, Thalassiosira pseudonana
MKVTTSIITLLFASCGAADVQRVLEDVTEPAVTTPAATPAPITPEPATPAPTICEGRNFYRDDDTGKCSNEATGGIYGTLIECCVAISGSDSCPYVDICNTLQPSPSPETNEPSAKPITAAPISSAPVSAAPVTSAPVAAPVETTSMTGPTTIVASIVSTNAPSSTNAPSSSLEAVVTRIPVETTNTASPTTTAASIVSTNAPSSSPEAVVTPRPTFRPSPKGTESNTFPASIASDVMFDPARSEPTFTPTSSSQPSSSEPTLSPSVSKEPTRYPTSSPSHSPTKSPSKSPSSSPTTSPSASPTETPTETPTESPTELPTLSPTEFPSLSPTLSPTWSPTGYPTLAPSPSPSISSAPSVSSAPSSSPSISSAPSVSSAPSKNFGFLPGRNEMPTISPTEDHYFFGKSHKSHKSKATKTLKVSKSGKSSKSSKSSGSRPLFGVSQLSEGIAAGYAKSSGRSSQQAVGSWMPVAAACILGALSFFLN

SEQ ID NO: 10 - protein, 501 aa, Thalassiosira pseudonana
MKVTTSIITLLFASCGAADVQRVLEDVTEPAVTTPAATPAPITPEPATPAPTICEGRNFYYDEETRKCSNEATGGIYGTLIDCCVAISGSVSCPYVDICNTLQPSPSPETNEPSAKPITAAPISSAPVSAAPVTSAPVAAPVETTSMTGPTTIVASIVSTNAPSLTNAPSSSLEAVVTRIPVETTNTASPTTTAASIVSTNAPSSSPEAVVTPRPTFRPSPEGTESNTSPASIASDVMFGPPKTSTPTSTPTSSSHPSSSEPTLSPSVSKEPTGYPTSSPSHSPTKSPSKSPSSSPTTSPSASPTETPTETPTESPTESPTESPTLSPTESPTLSPTESPSLSPTLSTTWSPTGYPTLAPSPSPSISSAPSVSSAPSSPPSISSAPSVSSAPSKNFGFLPGLTEMPTISPTEDHYFFGKSHKSHKSHKSKATKTLKVSKSGKSAKSSKSSGRRPLFGVSQLSEGIAVGYAKSSGRSSQQAVGSWMPVAAACILGALSFFLN

SEQ ID NO: 11 - skipped